SEQUENCE LISTING (43 sequences)

SEQ ID NO: 1 - DNA, 1091 bp, Arabidopsis thaliana
tcttcttctctacaacaaacaaacaaaagatctgtttcaatggcggatcgtgttaaaggtccatggagtcaagaagaagatgagcagctacgaaggatggttgagaaatacggaccgaggaattggtctgcgattagcaaatcgattccaggtcgatctggtaaatcgtgtagattacggtggtgtaatcagttatctccggaggttgagcatcgtcctttctcgccggaggaagatgagactattgtaaccgcccgtgctcagtttggtaacaagtgggcgacgattgctcgtcttcttaacggtcgtacggataacgccgttaaaaatcactggaactctacgcttaagaggaaatgcagcggaggtgtggcggttacgacggtgacggagacggaggaagatcaggatcggccgaagaagaggagatctgttagctttgattctgcttttgctccggtggatactggattgtacatgagtcctgagagtcctaacggaatcgatgttagtgattctagcacgattccgtcaccgtcgtctcctgttgctcagctgtttaaaccaatgccgatttccggcggttttacggtggttccgcagccgttaccggttgaaatgtcttcgtcttcggaggatccacctacttcgttgagtttgtcactacctggagctgagaacacgagttcgagccataacaataacaacaacgcgttgatgtttccgagatttgagagtcagatgaagattaatgtagaggagagaggaggaggaggagaaggacgtagaggtgagtttatgacggtggtgcaggagatgataaaagctgaagtgaggagttacatggcggaaatgcagaaaacaagtggtggattcgtcgtcggaggtttatacgaatccggcggcaatggtggttttagggattgtggaataataacacctaaggttgagtagttttggtttagggttaaaacttgaatcgattggggattttcaagagcattcatttttggggtttatggtaaaattaaaaacaaaaaaaaaatgtacagaggaattaaaatttctatggaataatcttaaatctaaaa

SEQ ID NO: 2 - DNA, 1419 bp, Zea mays
  misc_feature (84)..(84): n is a, c, g, or t
gggggcggccacgaaatatcagggccccaaattttagggttcccttaatgtgggttagttggaaggtggaaaaggtgtccaaanaagctatatntctttttttttttgggtaaatcgcgtgggagaaaatctttggtttggatttgacgggagctgctggccatggattgtagtagtagtcagtacagtagtatagcgagtccacctctcctctcccggcgggttcattctttacctaccacccaaccaagcccgaaacacgcgcgcccagagaaacgcaaggcttccttcagctttgactcagagccatatgaaagcagcatccgacgtcacatgcagagagcgccaaagtccaaggcgctacgtgacatctctgcatgaactcagctgttcaaacaatctttttattacattattacaatattaccacgtatataatcgtgtgtgtgataataataactatattactcgatcggtctgctgcgtccgtgtagactgcgaaataatcttgtcattgcgagcatccccgtattacctaaccagctgtactgtaccatatgcacgcagttgcttctcaactccgggcggcagtgtataagctgagcatatcacgagacacgtacgcaggccacacgtcctagctaggtcgtcgcagcactgcaggctgcagcagcgagcggccgggcagtatataagcatcgagcagggcttgccccgtagccacgacccacaccacacccactccgacattcggttgaattgaattggtgctgtatgtactgtgctgtgttagttgcatcggtttttgccggccggcaggccagcatggggaggtctccttgctgcaacgagaatggcctgaagaaggggccttggactcaggaggaagatgagaagctgctgcactacatccaaaagaacgggcatggcagttgggggaccctgcccaggctggccggtacatgaatgcatgcatgcccttctccgtccgatcgtctgtctgtctagctgtagctttgtatggattagctagctgatcagcgatggaggagctagctgaacatatatatatgcatgcagggctgaacaggtgcggcaagagctgccggctgcggtggaccaactacctgcggccggacatcaagcggggcaagttctcgcgggaggaggagcagaccatactccacctgcactccgtgctcggcaacaagtggggcgccatagccacgcacctcccaggccgcaccgacaacgagatcaagaacttttggaacacgcacctcaggaagcgcctcatacagatggggtttgaccccatgaggcaccgcccccgcaccgacttcttcgccgcgctgtcccagatcatatcgctctcagagctacgcgggcagagggcccggacacccgacccgccgcagc

SEQ ID NO: 3 - DNA, 819 bp, Glycine max
atggtgagagctccttgttgtgaaaagatgggtttgaagaagggtccttgggctaccgaggaagatcaaattctcacatcttacatccaaaaacatggccatggcaattggcgtgccctcccaaagcaagcaggccttctaaggtgtgggaaaagctgcagactccgctggattaactatttgaggcctgatatcaagagagggaatttcacaattgaagaagaagaaactatcattaagctgcatgagatgttgggaaacaggtggtcagcaattgcagccaaattgccaggaagaactgacaatgagataaaaaatgtttggcacaccaatttgaagaagaggctgctgaaatcagaccagtccaagccaagttccaaaagagccactaagccaaaaattaaacgttctgattccaattccagcatcataactcaatcagaacctgctcatcttagattcagagaaatggacacaacatcaacagcatgcaatacttctagtagtgatttttcatcagtcacggttggtgacagcaaaaatattatcaagagtgaagacatagagtctatggaaacaatgcctgtgatagatgagagcttttggtcagaagctgcaattgatgatgaaacccccaccatgtcatcacaatctttgatcacaatctcaaatgacatgccacttcaatacccttttgctaactatgaggaaactttccaacagagtcatgcttatgattcaaactttgatgatggcatggatttctggtatgacatattcaccaggaccaatgattcaatagaattgtcagagttctga

SEQ ID NO: 4 - DNA, 1275 bp, Gossypium hirsutum
tagaaattgttatacagttctagctaaggttcatttgaaagatacatacatacacacacatatatatatggggagatcaccatgttgtgaaaaggtagggttgaagaaaggtccatggaccccagaagaagatcaaaagctcttagcttacattgaacaacatggccatggaagctggcgtgccttgcctttaaaagctggtcttcatttctcaatatctttatacatatatatatgtttttgcttgctatatattaatttgatatatatatatatatatagggcttcaaagatgtggaaagagttgcagactgagatggattaactacttgagacctgatatcaaaagaggaaagttcagtttacaagaagaacagaccattattcaactccatgcccttcttggaaacaggtggtctgccatagctactcatttgccgaaaagaacagacaatgagatcaagaactactggaacacacatctaaagaaaaggctaaccaaaatggggatcgatcctgtcacccacaagcctaaaaccgatgcactcgggtccaccactggtaaccctatagatgctgctaaccttagtcacatggctcaatgggagagtgctcgtttagaagctgaagctagactggttcgtgagtccaagctagttccctcaaaccctcctcaaagcaaccacttcactgccgttgcgccttcgccgactccggcaactagaccgcaatgcctcgacgtactcaaagcatggcaaggtgtcgtctgcgggttattcactttcaacatggacaataacaacttacagtcccctacgtcaacgttgaacttcatggagaacaccacaacattgcctatgtcatcatcatcgtctgttaatggaatgtttaatgaaaactttggttggaactcatcgattaatccatgtgaaagtggggatattttgaaagttgaatatggcagtgatcaaattccagagttaaaggaaagattggatcatccaatggaattgcatgaaatggactgttcttcagagggtacatggtttcaagagttgtttggatttaatggtttgtgattctgcagaaggattcatcaaaggaaagaaaaagctatctggtttcatctttgaagttcacttaagtgtaggatttttattgacaagtgccttcacatattaccattaactgtaataataaaccttcaaattaataaatttaaaaaactcacaagggtttttggcctaaaaaaaaaaaaaaaaaaaaaaaaaaaaa

SEQ ID NO: 5 - DNA, 957 bp, Oryza sativa
ttactcgatcttggccatgcccatgatgcggctggcggcgcgcacgccgttgtcgggcgacgaccgaggatccacggcggcggagccggacatgtaattccggacctcgatccggatcatctcctgcatcatggccaagaactccgcgctgaacggcgccgatgggcgcggcggcggtgggggctgcagctggaacggcatctgaggcggctcttggcgtacacaagatggtgttggtggcggcatctgagacggtggaggcggctgcaactgtaccgcaggctccgacggggtggagggatcggggtgtgggtgatccaggccgggaagggagagagagagggagagcgaggtcgccggaggcggcgggggaggaggaggagggacgacgaccccgccggccctcgcgaccgggcggtagacgtgctgagggaggaggaggtgtgggggtgaggagggcatctgggaggggaggctatggtggctggagtcgctgaggtcggagccggaggggctcccgggggcggcggcggaggagagcgtcgggtggccgtcgctggttgtccgcttgagagggcggaggtcgtcggcgaggagagaagagtggtgcttgcgcttgagggtggagttccagtggttcttgatggcgttgtcggtgcggccggcgaggagcctggcgatggtggcccacttgttgccgaagcgggcgtgggcgcggaggatggtgtcgtcctcctcgggagtgaagggccggtgctccacctgcggcgacagctggttgcaccaccgcagccggcacgacttccccgacctccccgggatggacttgcttatcagcgaccagttgcgcgccccgtgccgccccaccagccgctgcagcgcctcgtcctcctccggactccacggcccctttatcctatccacatcccctccccctcctctccgacaagacgccat

SEQ ID NO: 6 - DNA, 1904 bp, Arabidopsis thaliana
aaggttgggaagaaggtgcaaatgacgtttagtgcgttactaacgtaagagacagttacacacaatttcaaaaacagttccaaaaatttgacacgtggcagtttgcttaatggacgacagttagttgtttgttgaggattacagcctggattttcgtcaatcaaaaactatatattataaataaatatgatttcattcacattccaattggcctaacactcttttgtcgtgttgtccatgtcagcaaattttatttttatttttccgaccacttacttttatcttgtgtggccgtttctttgtaacaagtttatgaaacagaatattaatcgtatctaatcatataaatgtagtttcccgtattgtgtaaatactatatgactttgatttggggcattcaaaaacgtaccattaacaaatagtcacagacaaaaaaaccatacaaaactgaaccacatttctataaacgatacaaatatttttatcccaaatttgtagcactaggcagtttgaacctttttcagttagaaagataaaccaaaaggaagtaaaaaatattaatttcatcgctcaaaactgttacaattactgtttaaccagtcataaattagttaatgtttttttttaaaaatgtgtaatcaaaattttactttcaagagtgccagctctatacttgcaattactttaaaatttaaaaatatacaaattttaaattatgtgtattgtattctcgagaaaaaaaaaaaaagaaatctatgaataattaaaacgtagcataaccgttacaagtatagtttcaaattcaacgaatcaaaatacttttttaaaagtagctaatacttttctcgtttcaaattaaatgtcgttttgcatattaatttttgtgtcatattaaacgtcgttttatattctatgcagattttatgtataaactttattggtcaaaagcgttacgaatatcgtttcaagttcaacgaatcaaaacatattttataaaaacatctaatacgttttccgtttcaaattagatgtcgttttgcattttaatttttgtgtcagtctaatgtcgttttatattttctacacatttatattttcaaaaatttcagttttatttttattgtttaaatatatcatccaatttgaaaataatacaatacaataacaaaaataacatttaaattaaacaaaaatatttttagaacactgtgatcaaattttatcctccaatatttcccgacccataaactttaaaccttatgtaacttcaaaaaaaaattcaaattagtcgttttgtattctacaaaataaaataaaaggaaaaaagtttcagagaaaagggtaaatttgcataacaaggaatacaggctggcgtgtccagtcaaaaaagtcagaaatctgcataaaaaactgtctttacccaaactgccaaacctaaccgttatcgtctcagatattattattatttttcaaataaacattcaaaacagctttctaatttttcacctcagattgtgcgaataatatcagagctctttccttgtaggtttccgtgattttgtcgctttaacttcccttcttttcccactctcttctcacgcgcgcgtgcttcccctttctctctttcgactgtatcacacgctccataaactaataaacagacgcaccgagatcacgacacgtgtacacctacagctgtatcccaaaactttcaaaaccgcttgcgtggaagaagcggtgcatgactttctccaccgtctctaccggttaccggttattatccatcctcctctctatctctctctctgttataaataccttttttctcctctcttcagtttcctccaccttcttcttcttcttcttcttctctacaacaaacaaacaaaagatctgtttcaatg

SEQ ID NO: 7 - DNA, 1901 bp, Arabidopsis thaliana
aaggttgggaagaaggtgcaaatgacgtttagtgcgttactaacgtaagagacagttacacacaatttcaaaaacagttccaaaaatttgacacgtggcagtttgcttaatggacgacagttagttgtttgttgaggattacagcctggattttcgtcaatcaaaaactatatattataaataaatatgatttcattcacattccaattggcctaacactcttttgtcgtgttgtccatgtcagcaaattttatttttatttttccgaccacttacttttatcttgtgtggccgtttctttgtaacaagtttatgaaacagaatattaatcgtatctaatcatataaatgtagtttcccgtattgtgtaaatactatatgactttgatttggggcattcaaaaacgtaccattaacaaatagtcacagacaaaaaaaccatacaaaactgaaccacatttctataaacgatacaaatatttttatcccaaatttgtagcactaggcagtttgaacctttttcagttagaaagataaaccaaaaggaagtaaaaaatattaatttcatcgctcaaaactgttacaattactgtttaaccagtcataaattagttaatgtttttttttaaaaatgtgtaatcaaaattttactttcaagagtgccagctctatacttgcaattactttaaaatttaaaaatatacaaattttaaattatgtgtattgtattctcgagaaaaaaaaaaaaagaaatctatgaataattaaaacgtagcataaccgttacaagtatagtttcaaattcaacgaatcaaaatacttttttaaaagtagctaatacttttctcgtttcaaattaaatgtcgttttgcatattaatttttgtgtcatattaaacgtcgttttatattctatgcagattttatgtataaactttattggtcaaaagcgttacgaatatcgtttcaagttcaacgaatcaaaacatattttataaaaacatctaatacgttttccgtttcaaattagatgtcgttttgcattttaatttttgtgtcagtctaatgtcgttttatattttctacacatttatattttcaaaaatttcagttttatttttattgtttaaatatatcatccaatttgaaaataatacaatacaataacaaaaataacatttaaattaaacaaaaatatttttagaacactgtgatcaaattttatcctccaatatttcccgacccataaactttaaaccttatgtaacttcaaaaaaaaattcaaattagtcgttttgtattctacaaaataaaataaaaggaaaaaagtttcagagaaaagggtaaatttgcataacaaggaatacaggctggcgtgtccagtcaaaaaagtcagaaatctgcataaaaaactgtctttacccaaactgccaaacctaaccgttatcgtctcagatattattattatttttcaaataaacattcaaaacagctttctaatttttcacctcagattgtgcgaataatatcagagctctttccttgtaggtttccgtgattttgtcgctttaacttcccttcttttcccactctcttctcacgcgcgcgtgcttcccctttctctctttcgactgtatcacacgctccataaactaataaacagacgcaccgagatcacgacacgtgtacacctacagctgtatcccaaaactttcaaaaccgcttgcgtggaagaagcggtgcatgactttctccaccgtctctaccggttaccggttattatccatcctcctctctatctctctctctgttataaataccttttttctcctctcttcagtttcctccaccttcttcttcttcttcttcttctctacaacaaacaaacaaaagatctgtttca

SEQ ID NO: 8 - DNA, 5009 bp, Artificial
  Translational fusion of the AtMyb77 Promoter and Initiator Methionine to a sequence comprising a Super- Ubiquitin Intron, a beta-Glucuronidase Gene, and a Nopaline Synthase Terminator
ggtaccaaggttgggaagaaggtgcaaatgacgtttagtgcgttactaacgtaagagacagttacacacaatttcaaaaacagttccaaaaatttgacacgtggcagtttgcttaatggacgacagttagttgtttgttgaggattacagcctggattttcgtcaatcaaaaactatatattataaataaatatgatttcattcacattccaattggcctaacactcttttgtcgtgttgtccatgtcagcaaattttatttttatttttccgaccacttacttttatcttgtgtggccgtttctttgtaacaagtttatgaaacagaatattaatcgtatctaatcatataaatgtagtttcccgtattgtgtaaatactatatgactttgatttggggcattcaaaaacgtaccattaacaaatagtcacagacaaaaaaaccatacaaaactgaaccacatttctataaacgatacaaatatttttatcccaaatttgtagcactaggcagtttgaacctttttcagttagaaagataaaccaaaaggaagtaaaaaatattaatttcatcgctcaaaactgttacaattactgtttaaccagtcataaattagttaatgtttttttttaaaaatgtgtaatcaaaattttactttcaagagtgccagctctatacttgcaattactttaaaatttaaaaatatacaaattttaaattatgtgtattgtattctcgagaaaaaaaaaaaaagaaatctatgaataattaaaacgtagcataaccgttacaagtatagtttcaaattcaacgaatcaaaatacttttttaaaagtagctaatacttttctcgtttcaaattaaatgtcgttttgcatattaatttttgtgtcatattaaacgtcgttttatattctatgcagattttatgtataaactttattggtcaaaagcgttacgaatatcgtttcaagttcaacgaatcaaaacatattttataaaaacatctaatacgttttccgtttcaaattagatgtcgttttgcattttaatttttgtgtcagtctaatgtcgttttatattttctacacatttatattttcaaaaatttcagttttatttttattgtttaaatatatcatccaatttgaaaataatacaatacaataacaaaaataacatttaaattaaacaaaaatatttttagaacactgtgatcaaattttatcctccaatatttcccgacccataaactttaaaccttatgtaacttcaaaaaaaaattcaaattagtcgttttgtattctacaaaataaaataaaaggaaaaaagtttcagagaaaagggtaaatttgcataacaaggaatacaggctggcgtgtccagtcaaaaaagtcagaaatctgcataaaaaactgtctttacccaaactgccaaacctaaccgttatcgtctcagatattattattatttttcaaataaacattcaaaacagctttctaatttttcacctcagattgtgcgaataatatcagagctctttccttgtaggtttccgtgattttgtcgctttaacttcccttcttttcccactctcttctcacgcgcgcgtgcttcccctttctctctttcgactgtatcacacgctccataaactaataaacagacgcaccgagatcacgacacgtgtacacctacagctgtatcccaaaactttcaaaaccgcttgcgtggaagaagcggtgcatgactttctccaccgtctctaccggttaccggttattatccatcctcctctctatctctctctctgttataaataccttttttctcctctcttcagtttcctccaccttcttcttcttcttcttcttctctacaacaaacaaacaaaagatctgtttcaatgaagcttgatatcgaattccgatattctcagagagcttttcattcaaaggtatggagttttgaagggctttactcttaacatttgtttttctttgtaaattgttaatggtggtttctgtgggggaagaatcttttgccaggtccttttgggtttcgcatgtttatttgggttatttttctcgactatggctgacattactagggctttcgtgctttcatctgtgttttcttcccttaataggtctgtctctctggaatatttaattttcgtatgtaagttatgagtagtcgctgtttgtaataggctcttgtctgtaaaggtttcagcaggtgtttgcgttttattgcgtcatgtgtttcagaaggcctttgcagattattgcgttgtactttaatattttgtctccaaccttgttatagtttccctcctttgatctcacaggaaccctttcttctttgagcattttcttgtggcgttctgtagtaatattttaattttgggcccgggttctgagggtaggtgattattcncagtgatgtgctttccctataaggtcctctatgtgtaagctgttagggtttgtgcgttactattgacatgtcacatgtcacatattttcttcctcttatccttcgaactgatggttctttttctaattcgtggattgctggtgccatattttatttctattgcaactgtattttagggtgtctctttctttttgatttcttgttaatatttgtgttcaggttgtaactatgggttgctagggtgtctgccctcttcttttgtgcttctttcgcagaatctgtccgttggtctgtatttgggtgatgaattatttattccttgaagtatctgtctaattagcttgtgatgatgtgcaggtatattcgttagtcatatttcaaggatccgggcccatggctccaaagaagaagagaaaggttgaagacccacgcatgttacgtcctgtagaaaccccaacccgtgaaatcaaaaaactcgacggcctgtgggcattcagtctggatcgcgaaaactgtggaattgatcagcgttggtgggaaagcgcgttacaagaaagccgggcaattgctgtgccaggcagttttaacgatcagttcgccgatgcagatattcgtaattatgcgggcaacgtctggtatcagcgcgaagtctttataccgaaaggttgggcaggccagcgtatcgtgctgcgtttcgatgcggtcactcattacggcaaagtgtgggtcaataatcaggaagtgatggagcatcagggcggctatacgccatttgaagccgatgtcacgccgtatgttattgccgggaaaagtgtacgtatcaccgtttgtgtgaacaacgaactgaactggcagactatcccgccgggaatggtgattaccgacgaaaacggcaagaaaaagcagtcttacttccatgatttctttaactatgccggaatccatcgcagcgtaatgctctacaccacgccgaacacctgggtggacgatatcaccgtggtgacgcatgtcgcgcaagactgtaaccacgcgtctgttgactggcaggtggtggccaatggtgatgtcagcgttgaactgcgtgatgcggatcaacaggtggttgcaactggacaaggcactagcgggactttgcaagtggtgaatccgcacctctggcaaccgggtgaaggttatctctatgaactgtgcgtcacagccaaaagccagacagagtgtgatatctacccgcttcgcgtcggcatccggtcagtggcagtgaagggcgaacagttcctgattaaccacaaaccgttctactttactggctttggtcgtcatgaagatgcggacttgcgtggcaaaggattcgataacgtgctgatggtgcacgaccacgcattaatggactggattggggccaactcctaccgtacctcgcattacccttacgctgaagagatgctcgactgggcagatgaacatggcatcgtggtgattgatgaaactgctgctgtcggctttaacctctctttaggcattggtttcgaagcgggcaacaagccgaaagaactgtacagcgaagaggcagtcaacggggaaactcagcaagcgcacttacaggcgattaaagagctgatagcgcgtgacaaaaaccacccaagcgtggtgatgtggagtattgccaacgaaccggatacccgtccgcaaggtgcacgggaatatttcgcgccactggcggaagcaacgcgtaaactcgacccgacgcgtccgatcacctgcgtcaatgtaatgttctgcgacgctcacaccgataccatcagcgatctctttgatgtgctgtgcctgaaccgttattacggatggtatgtccaaagcggcgatttggaaacggcagagaaggtactggaaaaagaacttctggcctggcaggagaaactgcatcagccgattatcatcaccgaatacggcgtggatacgttagccgggctgcactcaatgtacaccgacatgtggagtgaagagtatcagtgtgcatggctggatatgtatcaccgcgtctttgatcgcgtcagcgccgtcgtcggtgaacaggtatggaatttcgccgattttgcgacctcgcaaggcatattgcgcgttggcggtaacaagaaagggatcttcactcgcgaccgcaaaccgaagtcggcggcttttctgctgcaaaaacgctggactggcatgaacttcggtgaaaaaccgcagcagggaggcaaacaatgaatcaacaactctcctggcgcaccatcgtcggctacagcctcgggaattgctaccgagctccgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcg

SEQ ID NO: 9 - DNA, 5006 bp, Artificial
  Transciptional fusion of the AtMyb77 Promoter to a sequence comprising a Super-Ubiquitin Intron, a beta- Glucuronidase Gene,and a Nopaline Synthase Terminator
ggtaccaaggttgggaagaaggtgcaaatgacgtttagtgcgttactaacgtaagagacagttacacacaatttcaaaaacagttccaaaaatttgacacgtggcagtttgcttaatggacgacagttagttgtttgttgaggattacagcctggattttcgtcaatcaaaaactatatattataaataaatatgatttcattcacattccaattggcctaacactcttttgtcgtgttgtccatgtcagcaaattttatttttatttttccgaccacttacttttatcttgtgtggccgtttctttgtaacaagtttatgaaacagaatattaatcgtatctaatcatataaatgtagtttcccgtattgtgtaaatactatatgactttgatttggggcattcaaaaacgtaccattaacaaatagtcacagacaaaaaaaccatacaaaactgaaccacatttctataaacgatacaaatatttttatcccaaatttgtagcactaggcagtttgaacctttttcagttagaaagataaaccaaaaggaagtaaaaaatattaatttcatcgctcaaaactgttacaattactgtttaaccagtcataaattagttaatgtttttttttaaaaatgtgtaatcaaaattttactttcaagagtgccagctctatacttgcaattactttaaaatttaaaaatatacaaattttaaattatgtgtattgtattctcgagaaaaaaaaaaaaagaaatctatgaataattaaaacgtagcataaccgttacaagtatagtttcaaattcaacgaatcaaaatacttttttaaaagtagctaatacttttctcgtttcaaattaaatgtcgttttgcatattaatttttgtgtcatattaaacgtcgttttatattctatgcagattttatgtataaactttattggtcaaaagcgttacgaatatcgtttcaagttcaacgaatcaaaacatattttataaaaacatctaatacgttttccgtttcaaattagatgtcgttttgcattttaatttttgtgtcagtctaatgtcgttttatattttctacacatttatattttcaaaaatttcagttttatttttattgtttaaatatatcatccaatttgaaaataatacaatacaataacaaaaataacatttaaattaaacaaaaatatttttagaacactgtgatcaaattttatcctccaatatttcccgacccataaactttaaaccttatgtaacttcaaaaaaaaattcaaattagtcgttttgtattctacaaaataaaataaaaggaaaaaagtttcagagaaaagggtaaatttgcataacaaggaatacaggctggcgtgtccagtcaaaaaagtcagaaatctgcataaaaaactgtctttacccaaactgccaaacctaaccgttatcgtctcagatattattattatttttcaaataaacattcaaaacagctttctaatttttcacctcagattgtgcgaataatatcagagctctttccttgtaggtttccgtgattttgtcgctttaacttcccttcttttcccactctcttctcacgcgcgcgtgcttcccctttctctctttcgactgtatcacacgctccataaactaataaacagacgcaccgagatcacgacacgtgtacacctacagctgtatcccaaaactttcaaaaccgcttgcgtggaagaagcggtgcatgactttctccaccgtctctaccggttaccggttattatccatcctcctctctatctctctctctgttataaataccttttttctcctctcttcagtttcctccaccttcttcttcttcttcttcttctctacaacaaacaaacaaaagatctgtttcaaagcttgatatcgaattccgatattctcagagagcttttcattcaaaggtatggagttttgaagggctttactcttaacatttgtttttctttgtaaattgttaatggtggtttctgtgggggaagaatcttttgccaggtccttttgggtttcgcatgtttatttgggttatttttctcgactatggctgacattactagggctttcgtgctttcatctgtgttttcttcccttaataggtctgtctctctggaatatttaattttcgtatgtaagttatgagtagtcgctgtttgtaataggctcttgtctgtaaaggtttcagcaggtgtttgcgttttattgcgtcatgtgtttcagaaggcctttgcagattattgcgttgtactttaatattttgtctccaaccttgttatagtttccctcctttgatctcacaggaaccctttcttctttgagcattttcttgtggcgttctgtagtaatattttaattttgggcccgggttctgagggtaggtgattattcncagtgatgtgctttccctataaggtcctctatgtgtaagctgttagggtttgtgcgttactattgacatgtcacatgtcacatattttcttcctcttatccttcgaactgatggttctttttctaattcgtggattgctggtgccatattttatttctattgcaactgtattttagggtgtctctttctttttgatttcttgttaatatttgtgttcaggttgtaactatgggttgctagggtgtctgccctcttcttttgtgcttctttcgcagaatctgtccgttggtctgtatttgggtgatgaattatttattccttgaagtatctgtctaattagcttgtgatgatgtgcaggtatattcgttagtcatatttcaaggatccgggcccatggctccaaagaagaagagaaaggttgaagacccacgcatgttacgtcctgtagaaaccccaacccgtgaaatcaaaaaactcgacggcctgtgggcattcagtctggatcgcgaaaactgtggaattgatcagcgttggtgggaaagcgcgttacaagaaagccgggcaattgctgtgccaggcagttttaacgatcagttcgccgatgcagatattcgtaattatgcgggcaacgtctggtatcagcgcgaagtctttataccgaaaggttgggcaggccagcgtatcgtgctgcgtttcgatgcggtcactcattacggcaaagtgtgggtcaataatcaggaagtgatggagcatcagggcggctatacgccatttgaagccgatgtcacgccgtatgttattgccgggaaaagtgtacgtatcaccgtttgtgtgaacaacgaactgaactggcagactatcccgccgggaatggtgattaccgacgaaaacggcaagaaaaagcagtcttacttccatgatttctttaactatgccggaatccatcgcagcgtaatgctctacaccacgccgaacacctgggtggacgatatcaccgtggtgacgcatgtcgcgcaagactgtaaccacgcgtctgttgactggcaggtggtggccaatggtgatgtcagcgttgaactgcgtgatgcggatcaacaggtggttgcaactggacaaggcactagcgggactttgcaagtggtgaatccgcacctctggcaaccgggtgaaggttatctctatgaactgtgcgtcacagccaaaagccagacagagtgtgatatctacccgcttcgcgtcggcatccggtcagtggcagtgaagggcgaacagttcctgattaaccacaaaccgttctactttactggctttggtcgtcatgaagatgcggacttgcgtggcaaaggattcgataacgtgctgatggtgcacgaccacgcattaatggactggattggggccaactcctaccgtacctcgcattacccttacgctgaagagatgctcgactgggcagatgaacatggcatcgtggtgattgatgaaactgctgctgtcggctttaacctctctttaggcattggtttcgaagcgggcaacaagccgaaagaactgtacagcgaagaggcagtcaacggggaaactcagcaagcgcacttacaggcgattaaagagctgatagcgcgtgacaaaaaccacccaagcgtggtgatgtggagtattgccaacgaaccggatacccgtccgcaaggtgcacgggaatatttcgcgccactggcggaagcaacgcgtaaactcgacccgacgcgtccgatcacctgcgtcaatgtaatgttctgcgacgctcacaccgataccatcagcgatctctttgatgtgctgtgcctgaaccgttattacggatggtatgtccaaagcggcgatttggaaacggcagagaaggtactggaaaaagaacttctggcctggcaggagaaactgcatcagccgattatcatcaccgaatacggcgtggatacgttagccgggctgcactcaatgtacaccgacatgtggagtgaagagtatcagtgtgcatggctggatatgtatcaccgcgtctttgatcgcgtcagcgccgtcgtcggtgaacaggtatggaatttcgccgattttgcgacctcgcaaggcatattgcgcgttggcggtaacaagaaagggatcttcactcgcgaccgcaaaccgaagtcggcggcttttctgctgcaaaaacgctggactggcatgaacttcggtgaaaaaccgcagcagggaggcaaacaatgaatcaacaactctcctggcgcaccatcgtcggctacagcctcgggaattgctaccgagctccgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcg

SEQ ID NO: 10 - protein, 304 aa, Arabidopsis thaliana
MADRVKGPWSQEEDEQLRRMVEKYGPRNWSAISKSIPGRSGKSCRLRWCNQLSPEVEHRPFSPEEDETIVTARAQFGNKWATIARLLNGRTDNAVKNHWNSTLKRKCSGGVAVTTVTETEEDQDRPKKRRSVSFDSAFAPVDTGLYMSPESPNGIDVSDSSTIPSPSSPVAQLFKPMPISGGFTVVPQPLPVEMSSSSEDPPTSLSLSLPGAENTSSSHNNNNNALMFPRFESQMKINVEERGGGGEGRRGEFMTVVQEMIKAEVRSYMAEMQKTSGGFVVGGLYESGGNGGFRDCGIITPKVE

SEQ ID NO: 11 - protein, 298 aa, Drosophila melanogaster
MMMNAFIEPAQHHLASYGLRMSPNTTASNSNAQQQQQQQLEMTQQQQQQQQQQQQQQQQDQESAAATAAAYQNSGYGHFNSYASRDFLLGRREAEYGVAGSAGQASAAADSMLFSGFPAQAAELGSGFGQHPFHSHHHHHQMRMGMADAYAAGHPYNHHGNFPTAAVHHPVVHHPSHHAMSAMHPAGAGAFLRYMRHQPASSASSVKQEMQCLWIDPDQPGLVPPGGRKTCNKVFHSMHEIVTHLTVEHVGGPECTTHACFWVGCSRNGRPFKAKYKLVNHIRVHTGEKPFACPHPGC

SEQ ID NO: 12 - protein, 12 aa, Arabidopsis thaliana
LDLDLELRLGFA

SEQ ID NO: 13 - protein, 24 aa, Arabidopsis thaliana
EGGMEKRSQLLDLDLNLPPPSEQA

SEQ ID NO: 14 - DNA, 31 bp, Arabidopsis thaliana
tggtaccaaggttgggaagaaggtgcaaatg

SEQ ID NO: 15 - DNA, 30 bp, Arabidopsis thaliana
taagcttcattgaaacagatcttttgtttg

SEQ ID NO: 16 - DNA, 5994 bp, Artificial
  Plant Shuttle Vector pAKK1431
gatccttgaaatatgactaacgaatatacctgcacatcatcacaagctaattagacagatacttcaaggaataaataattcatcacccaaatacagaccaacggacagattctgcgaaagaagcacaaaagaagagggcagacaccctagcaacccatagttacaacctgaacacaaatattaacaagaaatcaaaaagaaagagacaccctaaaatacagttgcaatagaaataaaatatggcaccagcaatccacgaattagaaaaagaaccatcagttcgaaggataagaggaagaaaatatgtgacatgtgacatgtcaatagtaacgcacaaaccctaacagcttacacatagaggaccttatagggaaagcacatcactgngaataatcacctaccctcagaacccgggcccaaaattaaaatattactacagaacgccacaagaaaatgctcaaagaagaaagggttcctgtgagatcaaaggagggaaactataacaaggttggagacaaaatattaaagtacaacgcaataatctgcaaaggccttctgaaacacatgacgcaataaaacgcaaacacctgctgaaacctttacagacaagagcctattacaaacagcgactactcataacttacatacgaaaattaaatattccagagagacagacctattaagggaagaaaacacagatgaaagcacgaaagccctagtaatgtcagccatagtcgagaaaaataacccaaataaacatgcgaaacccaaaaggacctggcaaaagattcttcccccacagaaaccaccattaacaatttacaaagaaaaacaaatgttaagagtaaagcccttcaaaactccatacctttgaatgaaaagctctctgagaatatcggaattcgatatcaagcttatcgataccgtcgacctcgagggggggcccggtaccttaattaagcggccgcgtttaaacacgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgtgcgatcgcctgcaggcgcgccttaattaagagatccaattgcgatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaacgatcggggaaattcggagctcggtagcaattcccgaggctgtagccgacgatggtgcgccaggagagttgttgattcattgtttgcctccctgctgcggtttttcaccgaagttcatgccagtccagcgtttttgcagcagaaaagccgccgacttcggtttgcggtcgcgagtgaagatccctttcttgttaccgccaacgcgcaatatgccttgcgaggtcgcaaaatcggcgaaattccatacctgttcaccgacgacggcgctgacgcgatcaaagacgcggtgatacatatccagccatgcacactgatactcttcactccacatgtcggtgtacattgagtgcagcccggctaacgtatccacgccgtattcggtgatgataatcggctgatgcagtttctcctgccaggccagaagttctttttccagtaccttctctgccgtttccaaatcgccgctttggacataccatccgtaataacggttcaggcacagcacatcaaagagatcgctgatggtatcggtgtgagcgtcgcagaacattacattgacgcaggtgatcggacgcgtcgggtcgagtttacgcgttgcttccgccagtggcgcgaaatattcccgtgcaccttgcggacgggtatccggttcgttggcaatactccacatcaccacgcttgggtggtttttgtcacgcgctatcagctctttaatcgcctgtaagtgcgcttgctgagtttccccgttgactgcctcttcgctgtacagttctttcggcttgttgcccgcttcgaaaccaatgcctaaagagaggttaaagccgacagcagcagtttcatcaatcaccacgatgccatgttcatctgcccagtcgagcatctcttcagcgtaagggtaatgcgaggtacggtaggagttggccccaatccagtccattaatgcgtggtcgtgcaccatcagcacgttatcgaatcctttgccacgcaagtccgcatcttcatgacgaccaaagccagtaaagtagaacggtttgtggttaatcaggaactgttcgcccttcactgccactgaccggatgccgacgcgaagcgggtagatatcacactctgtctggcttttggctgtgacgcacagttcatagagataaccttcacccggttgccagaggtgcggattcaccacttgcaaagtcccgctagtgccttgtccagttgcaaccacctgttgatccgcatcacgcagttcaacgctgacatcaccattggccaccacctgccagtcaacagacgcgtggttacagtcttgcgcgacatgcgtcaccacggtgatatcgtccacccaggtgttcggcgtggtgtagagcattacgctgcgatggattccggcatagttaaagaaatcatggaagtaagactgctttttcttgccgttttcgtcggtaatcaccattcccggcgggatagtctgccagttcagttcgttgttcacacaaacggtgatacgtacacttttcccggcaataacatacggcgtgacatcggcttcaaatggcgtatagccgccctgatgctccatcacttcctgattattgacccacactttgccgtaatgagtgaccgcatcgaaacgcagcacgatacgctggcctgcccaacctttcggtataaagacttcgcgctgataccagacgttgcccgcataattacgaatatctgcatcggcgaactgatcgttaaaactgcctggcacagcaattgcccggctttcttgtaacgcgctttcccaccaacgctgatcaattccacagttttcgcgatccagactgaatgcccacaggccgtcgagttttttgatttcacgggttggggtttctacaggacgtaacatgcgtgggtcttcaacctttctcttcttctttggagccatgggcccg

SEQ ID NO: 17 - DNA, 11662 bp, Artificial
  Plant Transformation Vector AKK1472B
ggccgcggtaccgggccccccctcgaggtcgacggtatcgataagcttcacgctgccgcaagcactcagggcgcaagggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatcatgagcggagaattaagggagtcacgttatgacccccgccgatgacgcgggacaagccgttttacgtttggaactgacagaaccgcaacgttgaaggagccactcagccgcgggtttctggagtttaatgagctaagcacatacgtcagaaaccattattgcgcgttcaaaagtcgcctaaggtcactatcagctagcaaatatttcttgtcaaaaatgctccactgacgttccataaattcccctcggtatccaattagagtctcatattcactctcaatccagatctcgactctagggggatctaccatgagcccagaacgacgcccggccgacatccgccgtgccaccgaggcggacatgccggcggtctgcaccatcgtcaaccactacatcgagacaagcacggtcaacttccgtaccgagccgcaggaaccgcaggagtggacggacgacctcgtccgtctgcgggagcgctatccctggctcgtcgccgaggtggacggcgaggtcgccggcatcgcctacgcgggcccctggaaggcacgcaacgcctacgactggacggccgagtcgaccgtgtacgtctccccccgccaccagcggacgggactgggctccacgctctacacccacctgctgaagtccctggaggcacagggcttcaagagcgtggtcgctgtcatcgggctgcccaacgacccgagcgtgcgcatgcacgaggcgctcggatatgccccccgcggcatgctgcgggcggccggcttcaagcacgggaactggcatgacgtgggtttctggcagctggacttcagcctgccggtgccgccccgtccggtcctgcccgtcaccgagatctgatgacccctagaggatcagctcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattcctgcagcccctcgcggccgcgtttaaactatcagtgtttgacaggatatattggcgggtaaacctaagagaaaagagcgtttattagaataatcggatatttaaaagggcgtgaaaaggtttatccgttcgtccatttgtatgtgcatgccaaccacagggttccccagatctggcgccggccagcgagacgagcaagattggccgccgcccgaaacgatccgacagcgcgcccagcacaggtgcgcaggcaaattgcaccaacgcatacagcgccagcagaatgccatagtgggcggtgacgtcgttcgagtgaaccagatcgcgcaggaggcccggcagcaccggcataatcaggccgatgccgacagcgtcgagcgcgacagtgctcagaattacgatcaggggtatgttgggtttcacgtctggcctccggaccagcctccgctggtccgattgaacgcgcggattctttatcactgataagttggtggacatattatgtttatcagtgataaagtgtcaagcatgacaaagttgcagccgaatacagtgatccgtgccgccctggacctgttgaacgaggtcggcgtagacggtctgacgacacgcaaactggcggaacggttgggggttcagcagccggcgctttactggcacttcaggaacaagcgggcgctgctcgacgcactggccgaagccatgctggcggagaatcatacgcattcggtgccgagagccgacgacgactggcgctcatttctgatcgggaatgcccgcagcttcaggcaggcgctgctcgcctaccgcgatggcgcgcgcatccatgccggcacgcgaccgggcgcaccgcagatggaaacggccgacgcgcagcttcgcttcctctgcgaggcgggtttttcggccggggacgccgtcaatgcgctgatgacaatcagctacttcactgttggggccgtgcttgaggagcaggccggcgacagcgatgccggcgagcgcggcggcaccgttgaacaggctccgctctcgccgctgttgcgggccgcgatagacgccttcgacgaagccggtccggacgcagcgttcgagcagggactcgcggtgattgtcgatggattggcgaaaaggaggctcgttgtcaggaacgttgaaggaccgagaaagggtgacgattgatcaggaccgctgccggagcgcaacccactcactacagcagagccatgtagacaacatcccctccccctttccaccgcgtcagacgcccgtagcagcccgctacgggctttttcatgccctgccctagcgtccaagcctcacggccgcgctcggcctctctggcggccttctggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgcttttccgctgcataaccctgcttcggggtcattatagcgattttttcggtatatccatcctttttcgcacgatatacaggattttgccaaagggttcgtgtagactttccttggtgtatccaacggcgtcagccgggcaggataggtgaagtaggcccacccgcgagcgggtgttccttcttcactgtcccttattcgcacctggcggtgctcaacgggaatcctgctctgcgaggctggccggctaccgccggcgtaacagatgagggcaagcggatggctgatgaaaccaagccaaccaggaagggcagcccacctatcaaggtgtactgccttccagacgaacgaagagcgattgaggaaaaggcggcggcggccggcatgagcctgtcggcctacctgctggccgtcggccagggctacaaaatcacgggcgtcgtggactatgagcacgtccgcgagctggcccgcatcaatggcgacctgggccgcctgggcggcctgctgaaactctggctcaccgacgacccgcgcacggcgcggttcggtgatgccacgatcctcgccctgctggcgaagatcgaagagaagcaggacgagcttggcaaggtcatgatgggcgtggtccgcccgagggcagagccatgacttttttagccgctaaaacggccggggggtgcgcgtgattgccaagcacgtccccatgcgctccatcaagaagagcgacttcgcggagctggtgaagtacatcaccgacgagcaaggcaagaccgagcgcctttgcgacgctcaccgggctggttgccctcgccgctgggctggcggccgtctatggccctgcaaacgcgccagaaacgccgtcgaagccgtgtgcgagacaccgcggccaattctcatgtttgacagcttatcatcgataagctttaatgcggtagtttatcacagttaaattgctaacgcagtcaggcaccgtgtatgaaatctaacaatgcgctcatcgtcatcctcggcaccgtcaccctggatgctgtaggcataggcttggttatgccggtactgccgggcctcttgcgggatatcgtccattccgacagcatcgccagtcactatggcgtgctgctagcgctatatgcgttgatgcaatttctatgcgcacccgttctcggagcactgtccgaccgctttggccgccgcccagtcctgctcgcttcgctacttggagccactatcgactacgcgatcatggcgaccacacccgtcctgtggatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcactggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatcaattcttgcggagaactgtgaatgcgcaaaccaacccttggcagaacatatccatcgcgtccgccatctccagcagccgcacgcggcgcatctcgggcagcgttgggtcctggggccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggcccgctaacgcgggcctcccatccccccaggggctgcgcccctcggccgcgaacggcctcaccccaaaaatggcagcgctggcagtccttgccattgccgggatcggggcagtaacgggatgggcgatcagcccgagcgcgacgcccggaagcattgacgtgccgcaggtgctggcatcgacattcagcgaccaggtgccgggcagtgagggcggcggcctgggtggcggcctgcccttcacttcggccgtcggggcattcacggacttcatggcggggccggcaatttttaccttgggcattcttggcatagtggtcgcgggtgccgtgctcgtgttcgggggtgcgataaacccagcgaaccatttgaggtgataggtaagattataccgaggtatgaaaacgagaattggacctttacagaattactctatgaagcgccatatttaaaaagctaccaagacgaagaggatgaagaggatgaggaggcagattgccttgaatatattgacaatactgataagataatatatcttttatatagaagatatcgccgtatgtaaggatttcagggggcaaggcataggcagcgcgcttatcaatatatctatagaatgggcaaagcataaaaacttgcatggactaatgcttgaaacccaggacaataaccttatagcttgtaaattctatcataattgggtaatgactccaacttattgatagtgttttatgttcagataatgcccgatgactttgtcatgcagctccaccgattttgagaacgacagcgacttccgtcccagccgtgccaggtgctgcctcagattcaggttatgccgctcaattcgctgcgtatatcgcttgctgattacgtgcagctttcccttcaggcgggattcatacagcggccagccatccgtcatccatatcaccacgtcaaagggtgacagcaggctcataagacgccccagcgtcgccatagtgcgttcaccgaatacgtgcgcaacaaccgtcttccggagactgtcatacgcgtaaaacagccagcgctggcgcgatttagccccgacatagccccactgttcgtccatttccgcgcagacgatgacgtcactgcccggctgtatgcgcgaggttaccgactgcggcctgagttttttaagtgacgtaaaatcgtgttgaggccaacgcccataatgcgggctgttgcccggcatccaacgccattcatggccatatcaatgattttctggtgcgtaccgggttgagaagcggtgtaagtgaactgcagttgccatgttttacggcagtgagagcagagatagcgctgatgtccggcggtgcttttgccgttacgcaccaccccgtcagtagctgaacaggagggacagctgatagacacagaagccactggagcacctcaaaaacaccatcatacactaaatcagtaagttggcagcatcacccataattgtggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacctagatgtggcgcaacgatgccggcgacaagcaggagcgcaccgacttcttccgcatcaagtgttttggctctcaggccgaggcccacggcaagtatttgggcaaggggtcgctggtattcgtgcagggcaagattcggaataccaagtacgagaaggacggccagacggtctacgggaccgacttcattgccgataaggtggattatctggacaccaaggcaccaggcgggtcaaatcaggaataagggcacattgccccggcgtgagtcggggcaatcccgcaaggagggtgaatgaatcggacgtttgaccggaaggcatacaggcaagaactgatcgacgcggggttttccgccgaggatgccgaaaccatcgcaagccgcaccgtcatgcgtgcgccccgcgaaaccttccagtccgtcggctcgatggtccagcaagctacggccaagatcgagcgcgacagcgtgcaactggctccccctgccctgcccgcgccatcggccgccgtggagcgttcgcgtcgtctcgaacaggaggcggcaggtttggcgaagtcgatgaccatcgacacgcgaggaactatgacgaccaagaagcgaaaaaccgccggcgaggacctggcaaaacaggtcagcgaggccaagcaggccgcgttgctgaaacacacgaagcagcagatcaaggaaatgcagctttccttgttcgatattgcgccgtggccggacacgatgcgagcgatgccaaacgacacggcccgctctgccctgttcaccacgcgcaacaagaaaatcccgcgcgaggcgctgcaaaacaaggtcattttccacgtcaacaaggacgtgaagatcacctacaccggcgtcgagctgcgggccgacgatgacgaactggtgtggcagcaggtgttggagtacgcgaagcgcacccctatcggcgagccgatcaccttcacgttctacgagctttgccaggacctgggctggtcgatcaatggccggtattacacgaaggccgaggaatgcctgtcgcgcctacaggcgacggcgatgggcttcacgtccgaccgcgttgggcacctggaatcggtgtcgctgctgcaccgcttccgcgtcctggaccgtggcaagaaaacgtcccgttgccaggtcctgatcgacgaggaaatcgtcgtgctgtttgctggcgaccactacacgaaattcatatgggagaagtaccgcaagctgtcgccgacggcccgacggatgttcgactatttcagctcgcaccgggagccgtacccgctcaagctggaaaccttccgcctcatgtgcggatcggattccacccgcgtgaagaagtggcgcgagcaggtcggcgaagcctgcgaagagttgcgaggcagcggcctggtggaacacgcctgggtcaatgatgacctggtgcattgcaaacgctagggccttgtggggtcagttccggctgggggttcagcagccagcgctttactggcatttcaggaacaagcgggcactgctcgacgcacttgcttcgctcagtatcgctcgggacgcacggcgcgctctacgaactgccgataaacagaggattaaaattgacaattgtgattaaggctcagattcgacggcttggagcggccgacgtgcaggatttccgcgagatccgattgtcggccctgaagaaagctccagagatgttcgggtccgtttacgagcacgaggagaaaaagcccatggaggcgttcgctgaacggttgcgagatgccgtggcattcggcgcctacatcgacggcgagatcattgggctgtcggtcttcaaacaggaggacggccccaaggacgctcacaaggcgcatctgtccggcgttttcgtggagcccgaacagcgaggccgaggggtcgccggtatgctgctgcgggcgttgccggcgggtttattgctcgtgatgatcgtccgacagattccaacgggaatctggtggatgcgcatcttcatcctcggcgcacttaatatttcgctattctggagcttgttgtttatttcggtctaccgcctgccgggcggggtcgcggcgacggtaggcgctgtgcagccgctgatggtcgtgttcatctctgccgctctgctaggtagcccgatacgattgatggcggtcctgggggctatttgcggaactgcgggcgtggcgctgttggtgttgacaccaaacgcagcgctagatcctgtcggcgtcgcagcgggcctggcgggggcggtttccatggcgttcggaaccgtgctgacccgcaagtggcaacctcccgtgcctctgctcacctttaccgcctggcaactggcggccggaggacttctgctcgttccagtagctttagtgtttgatccgccaatcccgatgcctacaggaaccaatgttctcggcctggcgtggctcggcctgatcggagcgggtttaacctacttcctttggttccgggggatctcgcgactcgaacctacagttgtttccttactgggctttctcagccccagatctggggtcgatcagccggggatgcatcaggccgacagtcggaacttcgggtccccgacctgtaccattcggtgagcaatggataggggagttgatatcgtcaacgttcacttctaaagaaatagcgccactcagcttcctcagcggctttatccagcgatttcctattatgtcggcatagttctcaagatcgacagcctgtcacggttaagcgagaaatgaataagaaggctgataattcggatctctgcgagggagatgatatttgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattggcgatcgcctgcaggcgcgccttaattaagc

SEQ ID NO: 18 - DNA, 27 bp, Arabidopsis thaliana
cgaaagtggggttagggtatgtgaaag

SEQ ID NO: 19 - DNA, 28 bp, Arabidopsis thaliana
tttaactcaacactcaagaaacaagtag

SEQ ID NO: 20 - DNA, 24 bp, Arabidopsis thaliana
acgttcggattcgcgtctacaccg

SEQ ID NO: 21 - DNA, 25 bp, Arabidopsis thaliana
tcatggcttgtgaggagggcattgg

SEQ ID NO: 22 - DNA, 24 bp, Arabidopsis thaliana
atggcttttttgaggagtttcttg

SEQ ID NO: 23 - DNA, 25 bp, Arabidopsis thaliana
tcattgtatctgagatgtgactgtg

SEQ ID NO: 24 - DNA, 26 bp, Arabidopsis thaliana
caggctaaggtgatgccaccaacaag

SEQ ID NO: 25 - DNA, 27 bp, Arabidopsis thaliana
tcatagacccaagagaatgtagtagag

SEQ ID NO: 26 - DNA, 27 bp, Arabidopsis thaliana
cttgtcattgaggaacgagttaaaaac

SEQ ID NO: 27 - DNA, 27 bp, Arabidopsis thaliana
atgtaagtccccaacatcggaacaaac

SEQ ID NO: 28 - DNA, 26 bp, Arabidopsis thaliana
tgcctctggaacttcttacggtttag

SEQ ID NO: 29 - DNA, 26 bp, Arabidopsis thaliana
ggctgagttatcatcataatgacctc

SEQ ID NO: 30 - DNA, 26 bp, Arabidopsis thaliana
cttcaacctgcaaggacctacgcgag

SEQ ID NO: 31 - DNA, 27 bp, Arabidopsis thaliana
atccgttagtagtaatgatttgcctgg

SEQ ID NO: 32 - DNA, 28 bp, Arabidopsis thaliana
taacagggttgaggtagctccagtggtg

SEQ ID NO: 33 - DNA, 28 bp, Arabidopsis thaliana
ctattccttgccatgttttctaggtgag

SEQ ID NO: 34 - DNA, 25 bp, Arabidopsis thaliana
atggaagtcaccaatgggcttaacc

SEQ ID NO: 35 - DNA, 26 bp, Arabidopsis thaliana
tcataaggcagtaggagcttcggatc

SEQ ID NO: 36 - DNA, 33 bp, Artificial
  Sequence from Arabidopsis AtMyb77 Promoter and HindIII restriction enzyme site
taagctttgaaacagatcttttgtttgtgaaac

SEQ ID NO: 37 - DNA, 31 bp, Artificial
  Sequence from Arabidopsis AtMyb77 cDNA and NdeI restriction enzyme site
catatgatggcggatcgtgttaaaggtccat

SEQ ID NO: 38 - DNA, 32 bp, Artificial
  Sequence from Arabidopsis AtMyb77 cDNA and SacI restriction enzyme site
gagctcctcaaccttaggtgttattactccac

SEQ ID NO: 39 - DNA, 21 bp, Arabidopsis thaliana
  misc_feature (16)..(16): n is a, c, g, or t
aaccwaccaaccaacngaacc

SEQ ID NO: 40 - DNA, 24 bp, Arabidopsis thaliana
cagttgcgttrcacggttactgac

SEQ ID NO: 41 - DNA, 21 bp, Artificial
  Mutated Sequence from Arabidopsis MRE element
agccwgccagccagcngagcc

SEQ ID NO: 42 - DNA, 24 bp, Artificial
  Mutated Sequence from Arabidopsis MYBcore element
cagtagcgtarcacggtaactgac

SEQ ID NO: 43 - DNA, 27 bp, Arabidopsis thaliana
ataaagagccgccactaaaataagctt

(19) United States
(12) Patent Application Publication
(10) Pub. No.: US 2008/0196124 A1
(43) Pub. Date: Aug. 14, 2008

(54) Plant Growth Hormone Regulated Transcription Factors and Promoters Thereof

(75) Inventors: Daniel P. Schachtman, St. Louis, MO (US); Ryoung Shin, St. Louis, MO (US)

Correspondence Address: THOMPSON COBURN, LLP, ONE US BANK PLAZA, SUITE 3500, ST LOUIS, MO, 63101, US

(73) Assignee: DONALD DANFORTH PLANT SCIENCE CENTER, St. Louis, MO (US)

(21) Appl. No.: 11/741,223

(22) Filed: Apr. 27, 2007

Related U.S. Application Data
(60) Provisional application No. 60/745,786, filed on Apr. 27, 2006.

(51) Int. Cl. C12N 15/29; C12N 5/10

(52) U.S. Cl. 800/287; 800/278; 800/294; 800/291; 435/419; 435/418

(57) ABSTRACT

Methods for obtaining plants with decreased responses to auxins by reducing exprssion of endogenous MYB77 genes in plants are disclosed. Also disclosed are methods of using plant MYB77 transcriptional promoter sequences to drive expression of heterologous genes in select plant cells at select times in plant development. Methods of obtaining plants with decreased lateral root formation when grown under nutrient deficient conditions by reducing expression of endogenous MYB77 and transgenic plants with reduced expression of endogenous MYB77 genes in are also provided.

### CROSS-REFERENCE TO RELATED APPLICATIONS

This application claims priority to the Apr. 27, 2006 filing date of U.S. Provisional Patent Application No. 60/745,786, which is herein incorporated by reference in its entirety.

### STATEMENT REGARDING FEDERALLY SPONSORED RESEARCH OR DEVELOPMENT

Not Applicable.

### INCORPORATION OF SEQUENCE LISTING

A paper copy of the Sequence Listing and a computer readable form of the sequence listing are herein incorporated by reference. This Sequence Listing consists of SEQ ID NOs:1-43.

### FIELD OF THE INVENTION

The present invention relates to methods for obtaining plants with decreased responses to auxins. The invention further relates to use of plant transcriptional promoter sequences that are useful for driving expression of heterologous genes in select plant cells at select times in plant development. The invention also discloses methods of obtaining plants with decreased lateral root formation when grown under nutrient deficient conditions.

### BACKGROUND TO THE INVENTION

Auxin is a well known regulator of plant growth (Weijers and Jürgens 2004) and synthetic auxins have been used as highly effective herbicides (Lym and Moxness 1989), illustrating that low concentrations of auxin can promote plant growth while higher concentrations of auxin can be phytotoxic. Auxin is known to play an important role in lateral root growth (López-Bucio et al. 2003; Montiel et al. 2004). Methods of controlling a plant's response to both endogenous and exogenous auxins are useful in that they can provide for methods of altering plant growth patterns. For example, controlling a plant's response to endogenous auxin would permit control of lateral root growth, initiation or formation. Alternatively, controlling a plant's response to exogenous auxin can provide for methods of reducing a plant's sensitivity to auxin herbicides. Plants that are resistant to auxin herbicides are useful in agricultural settings where the resistant crop plant and susceptible weeds are both exposed to the auxin herbicide. Transgenes that provide for decreased herbicide sensitivity also find utility as selectable or scoreable markers in plant transformation experiments.

Myb transcription factors are found in plants and animals. The R2R3 type of Myb transcription factors are found only in plants (Petroni et al. 2002). The first Myb transcription factors isolated from plants were found in maize and were shown to be involved in anthocyanin production. These Myb transcription factors have been shown to interact with another class of transcription factors to impart combinatorial control of secondary metabolite production (Cone et al. 1986). The R2R3 Myb transcription factor family in Arabidopsis comprises a large gene family in plants with 125 members (Stracke et al. 2001). The functional diversity of processes that these transcription factors regulate ranges from the synthesis of secondary metabolites to plant development (Oppenheimer et al. 1991; Lee and Schiefelbein 1999; Nesi et al. 2001).

Previous gene expression studies on the R2R3 Myb transcription factor family in Arabidopsis showed that the addition of auxin increases the expression of AtMYB7, AtMYB9, AtMYB13, AtMYB14, AtMYB30, AtMYB60, AtMYB77, AtMYB91 and AtMYB93 (Kranz et al. 1998). Kranz et al. also indicate that AtMYB77 (referred to herein as AtMyb77) is one of several MYB genes that display a modest increase in expression under growth conditions where nitrogen is present but sucrose absent.

AtMyb77 expression has also been shown to be decreased by potassium (K+) deprivation; under these conditions a large decrease in lateral root growth is measured (Armengaud et al. 2004; Shin and Schachtman, 2004).

U.S. patent application Ser. No. 10/632,436, incorporated by reference herein in its entirety, identifies AtMyb77 as one of 7 transcription factors whose expression is increased by cold treatment in Table 2 of that patent application.

European Patent Application No. 05007595.1 for “Yield Related Genes” discloses use of 35 transcription factors from Arabidopis including members of the MYB family as genes involved in sugar sensing that can allegedly be used to improve plant yield. A SEQ ID NO:7 disclosed in this EU application appears to be AtMyb77. Table 3 of this patent application indicates that overexpression of the SEQ ID NO:7 of that application in transgenic plants results in reduced germination on glucose containing media.

International Patent Application No. WO2005047516 for “Plant Transcriptional Regulators” lists AtMyb77 (internal designator is G207; SEQ ID NO:910 of that application) in Table 4 along with over 500 other plant transcription factors as a “polypeptides of the invention”. Other tables in the application identify genes from other plant species that are “paralogs” or “orthologs” to AtMyb77. This patent application is directed to compositions and methods that purportedly modify the phenotype of a plant, alleging that altered carbon/nitrogen balance sensing, improved nitrogen uptake or assimilation efficiency, improved growth or survival of plants under conditions of nitrogen limitation, increased tolerance to drought or other abiotic stress, and/or increased tolerance to shade is attained.

International Patent Application No. WO2004035798 and U.S. patent application Ser. No. 10/531,475 for “Identification Of Novel E2f Target Genes And Use Thereof” allegedly discloses methods for altering one or more plant characteristics, including ranging from altered development, altered plant growth, increased plant yield and/or biomass, biochemistry, physiology, architecture, metabolism, survival capacity or stress tolerance by modifying expression of one or more of the 2,755 disclosed genes or proteins encoded by these genes. Among the 2,755 disclosed genes, this patent application identifies At3g50060 (AtMyb77) in Table 5 as one of over approximately 600 Arabidopsis genes that are 1.3 times (1/ratio) or more repressed in E2Fa/Dpa overexpressing transgenic plants.

International Patent Application No. WO2002016655 identifies SEQ ID NO:1-2703 as potentially useful coding regions for increasing or decreasing the stress tolerance of plants and SEQ ID NO:2704-5379 as potentially useful regulatory sequences for controlling expression of genes. A DNA fragment corresponding to 2,000 nucleotides of the sequence immediately 5′ to the translation initiation site of the AtMyb77 gene is disclosed as SEQ ID NO:4367 of that application. This same sequence is disclosed in International Patent Application No. WO2003000898 as SEQ ID NO:2609 of that application. International Patent Application No. WO2003000898 identifies SEQ ID NO:2137-2661 and SEQ ID NO:4738-6813 as plant nucleotide sequences that alter transcription of operatively linked plant genes in a plant cell after pathogen infection. International Patent Application No. WO2003000898 also identifies SEQ ID NO:1-953, SEQ ID NO:1954-1966, SEQ ID NO:2000-2129, and SEQ ID NO:2662-4737 as plant protein sequences encoded by genes that are useful in practicing methods of identifying plant genes involved in plant pathogen resistance or response to plant pathogens.

A first object of this invention is to provide methods of obtaining plants with decreased responses to either endogenous or exogenous auxin, thus conferring useful growth characteristics or resistance to auxin-type herbicides. A second object of this invention is to provide methods for obtaining plants with reduced lateral root formation and, more specifically, plants that display reduced lateral root formation when grown under nutrient deprived growth conditions. A third object of this invention is to provide methods for expressing sequences of interest in auxin responsive plant root cells. Plant cells and plants produced by the aforementioned methods of this invention are also provided.

### SUMMARY OF THE INVENTION

Methods of Obtaining Plants with a Decreased Auxin Response

The instant invention first provides for a method of obtaining a plant with a decreased response to auxin, comprising the steps of: (a) introducing an agent capable of reducing expression of at least one endogenous MYB77 gene in a plant cell; (b) regenerating a plant from said plant cell of step (a); and (c) identifying a regenerated plant from step (b) wherein said agent has reduced expression of said MYB77 gene in said regenerated plant by an amount sufficient to decrease a response of said regenerated plant to auxin.

The introduced agent can be any one of:
i) a compound that effects an insertion, deletion, or point mutation in an endogenous Myb77 gene;
ii) ionizing radiation or ultraviolet light that effects an insertion, deletion, or point mutation in an endogenous MYB77 gene;
iii) a DNA fragment such as a T-DNA or transposon that is capable of insertion into a plant cell's genome (the T-DNA can be introduced into said plant cells with any one of Agrobacterium tumefaciens, Agrobacterium rhizogenes, Rhizobium, and Sinorhizobium);
iv) a transgene comprising any one of a fragment of the MYB77 gene, a sequence for producing a small interfering RNA (siRNA) capable of reducing expression of at least one endogenous MYB77 gene or a dominant negative mutant allele of said MYB77 gene. The dominant negative form of the MYB77 gene may comprise a translational fusion of at least one DNA binding domain of the MYB77 gene and a transcriptional repressor domain, such as an Engrailed protein transcriptional repressor domain, an EAR protein transcriptional repressor domain, or an IAA/AUX domain I transcriptional repressor domain.

This method is useful for reducing the response of a plant cell or a plant when the auxin is:
i) produced by a plant cell, a group of plant cells, one or more cells of a whole plant, or any combination thereof;
ii) auxin provided by an external source such as a herbicidal formulation that is applied to the plant cell or plants by watering, spraying, atomizing, broadcasting, dusting, foaming, spreading, ultra-low volume method, treating soil, treating growth media or treating seeds;
iii) a naturally occurring auxin or an auxin precursor such as indoleacetic acid, indolebutyric acid, indole-3-acetyl-myo-inositol ester, esters thereof, and amide amino acid conjugates thereof; and/or
iii) a synthetic auxin such as 2,4-dichloroacetic acid, naphthaleneacetic acid, clomeprop, 2,4,5-trichlorophenoxy acetic acid, 2,4-dichlorophenoxy butyric acid, dichlorprop, dichlorprop-P, monochlorophenoxy acetic acid, monochlorophenoxy acetic acid thioethyl, monochlorophenoxy butyric acid, mecoprop, mecoprop-P, chloramben, dicamba, 2,3,6-trichlorobenzoate, tricamba, quinchlorac, quinmerac, clopyralid, fluoroxypyr, picloram, trichlopyr, benazolin, chloramben, dicamba, 2-fluoroindole-3-acetic acid, 4-fluoroindole-3-acetic acid, 6-fluoroindole-3-acetic acid, 7-fluoroindole-3-acetic acid, 2-bromoindole-3-acetic acid, 4-bromoindole-3-acetic acid, 6-bromoindole-3-acetic acid, 7-bromoindole-3-acetic acid, 2-iodoindole-3-acetic acid, 4-iodoindole-3-acetic acid, 5-iodoindole-3-acetic acid, 6-iodoindole-3-acetic acid, 7-iodoindole-3-acetic acid, salts thereof, esters thereof, and amides thereof.

This method is also useful for reducing an auxin response such as inhibition of lateral root formation that occurs when sufficient auxin is provided by an external source.

Identification of plants with reduced expression of the MYB77 gene can be accomplished by any number of techniques. For example, MYB77 gene expression can be assayed by methods such as MYB77 sequence specific DNA binding assays, enzyme-linked MYB77 immunoassays, assays based on MYB77 RNA hybridization and assays employing reverse-transcriptase polymerase chain reaction to detect MYB77 sequences. It is noted herein that reduced expression of MYB77 can result in decreased expression of certain RNA sequences or protein sequences that require MYB77 for expression. For example, expression of auxin induced gene sequences or proteins, such as those for the AXR1, IAA19, IAA1, IAA28, SAUR-AC1, ARF7 and AUX1 genes, is shown herein to be decreased in plants with reduced MYB77 expression relative to wild-type plants upon auxin exposure. Consequently, plants or plant cells with reduced expression of MYB77 can be identified by assaying for reductions in expression of genes regulated by the MYB77 gene (i.e., AXR1, IAA19, IAA1, IAA28, SAUR-AC1, ARF7 and/or AUX1 genes) in auxin exposed plants by methods such as an enzyme activity assay, a non-enzymatic biochemical activity assay, an immunoassay, an enzyme-linked immunoassay, an assay based on detection by RNA hybridization, and an assay based on detection by reverse-transcriptase polymerase chain reaction. In this case, the assay would be aimed at detecting the gene regulated by MYB77.

Identification of plants with reduced expression of the MYB77 gene may also be confirmed by assaying a biological effect of this reduced expression. For example, decreased lateral root formation is a biological response that occurs in plants after exposure to sufficient concentrations of exogenous auxin. Plants subjected to these methods can be assayed for increased lateral root formation relative to control plants (i.e., plants not subjected to the methods of the invention) after auxin exposure to identify plant or plant cells with reduced expression of MYB77.

This method is found to be useful in a variety of different types of plants when the expression of the appropriate MYB77 gene is reduced in that plant. When the plant cell is an Arabidopsis thaliana plant, the MYB77 gene may encode a cDNA sequence selected from the group of cDNA sequences that are essentially identical to SEQ ID NO:1. When the plant is a Zea mays plant, the MYB77 gene may encode a cDNA essentially identical to SEQ ID NO:2. When the plant is a Glycine max plant, the MYB77 gene encodes a cDNA essentially identical to SEQ ID NO:3. When the plant is a Gossypium hirsutum plant, the MYB77 gene encodes a cDNA essentially identical to SEQ ID NO:4. When the plant is an Oryzae species plant, the MYB77 gene encodes a cDNA essentially identical to SEQ ID NO:5.

### Methods of Expressing Sequences of Interest in an Auxin Responsive Plant Root Cell

The instant invention also provides a method for obtaining a transgenic plant expressing a sequence of interest in an auxin-responsive plant root cell, comprising the steps of: (a) constructing a DNA molecule comprising a functional AtMyb77 promoter fragment that is operably linked to said sequence of interest; (b) integrating said DNA molecule of step (a) into a plant cell's genome to obtain a transgenic plant cell; (c) regenerating a transgenic plant from said transgenic plant cell of step (b); and (d) confirming expression of said sequence of interest in an auxin-responsive plant cell of said transgenic plant of step (c).

In this method the sequence of interest can be any one of:
i) a gene encoding a reporter protein such as a beta-glucuronidase protein, a green fluorescent protein, a beta-galactosidase protein, a luciferase protein derived from a luc gene, a luciferase protein derived from a lux gene, a sialidase protein, streptomycin phosphotransferase protein, a nopaline synthase protein, an octopine synthase protein or a chloramphenicol acetyl transferase protein;
ii) a sequence capable of reducing a response to auxin in a plant root cell such as a sequence comprising an antisense sequence, a silencing sequence, or a sequence encoding a small interfering RNA designed to decrease expression of a gene encoding a protein such as a TIR1 protein, an AXR1 protein, an IAA19 protein, an IAA1 protein, an IAA28 protein, a SAUR-AC1 protein, a PIN1 protein, an ARF7 protein and an AUX1 protein; or
iii) any other coding sequence, antisense sequence, silencing sequence or sequence for producing an siRNA sequence that could confer a useful trait or characteristic when expressed in an auxin responsive plant cell.

The functional AtMyb77 promoter fragment used in this method may comprise SEQ ID NO:6 or SEQ ID NO:7. This AtMyb77 promoter fragment can be operably linked to a beta-glucuronidase gene which is operably linked to a polyadenylation region. The AtMyb77 promoter of SEQ ID NO:6 can be operably linked as a translational fusion to a beta-glucuronidase coding sequence and a polyadenylation region as shown in SEQ ID NO:8. Alternatively, the AtMyb77 promoter of SEQ ID NO:7 can be operably linked as a transcriptional fusion to a beta-glucuronidase coding sequence and a polyadenylation region as shown in SEQ ID NO:9.

Expression of said sequence of interest can be confirmed by any number of techniques. For example, expression can be assayed by methods such as an enzyme activity assay, a non-enzymatic biochemical activity assay, an immunoassay, an enzyme-linked immunoassay, an assay based on detection by RNA hybridization, and an assay based on detection by reverse-transcriptase polymerase chain reaction. It is noted herein that expression of the sequence of interest may in some instances result in an increase in expression of a desired RNA sequence or protein sequence that is encoded by the sequence of interest. However, in other instances where the sequence of interest encodes a fragment of a gene, an antisense sequence of a gene, or a sequence for producing a small inhibiting RNA directed against an endogenous plant gene, expression of the sequence of interest may result in a decrease in the expression of an endogenous plant gene (i.e., the “target” of the fragment of a gene, an antisense sequence of a gene, or a small inhibiting RNA). In these instances, expression of the sequence of interest can be indirectly assayed by identifying plants or plant cells with decreased expression of the endogenous plant gene targeted for decreased expression by the sequence of interest. The endogenous plant gene's expression can be assayed by methods such as enzyme activity-based determination assays, biochemical activity-based assays, an enzyme-linked immunoassays, an RNA hybridization based assays, and reverse-transcriptase polymerase chain reaction-based assays.

Expression of the sequence of interest can also be confirmed by assaying a biological effect of this expression. When the sequence of interest modulates an auxin response pathway (i.e., either induces or represses an auxin response), expression can be confirmed by analyzing the relevant auxin response. For example, decreased lateral root formation is a biological response that occurs in plants after exposure to sufficient concentrations of exogenous auxin. Plants subjected to these methods can be assayed for increased lateral root formation relative to control plants (i.e., plants not subjected to the methods of the invention) after auxin exposure to identify plant or plant cells that express the sequence of interest.

Methods for Obtaining Plants with Decreased Lateral Root Formation in Potassium Deficient Growth Conditions

Decreased lateral root formation is observed in many plants in response to potassium deficient growth conditions. Without being limited by theory, it is believed that reduced lateral root formation under conditions of potassium deficiency is a desirable growth characteristic in that it may more usefully channel or conserve plant photosynthate use by a plant grown under these potassium deficient conditions. It is understood that a number of different growth conditions can comprise or result in potassium deficient growth conditions. For example, the soil or the media that the plants are grown in may simply be deficient in potassium. Other growth conditions that can result in potassium deficient growth conditions are conditions that result in decreased potassium availability. For example, conditions of drought or water deficit can result in potassium deficient growth conditions by decreasing potassium availability (“Potassium Reduces Stress from Drought, Cool Soils and Compaction” In Better Crops with Plant Food. Vol. 82 (1998, No. 3):34-36). Without being limited by theory, reduced lateral root growth under conditions of drought-induced potassium deficits may lead to increased primary root growth, permitting deeper penetration of the primary root into the soil to obtain moisture. Reduced lateral root growth under conditions of drought-induced potassium deficits is thus another desirable growth characteristic achieved by the practice of this invention. Other non-limiting examples of growth conditions that result in reduced potassium availability include decreased soil oxygenation (especially when the soil is saturated with water), conditions of low soil temperature, conditions of potassium stratification near the soil surface, conditions of soil compaction and/or conditions of “no-till” or “ridge till” cultivation (Rehm, G., and Schmitt, M. (1997) Potassium for Crop Production, University of Minnesota Extension Service; and “Potassium Reduces Stress from Drought, Cool Soils and Compaction” In Better Crops with Plant Food. Vol. 82 (1998, No. 3):34-36). This invention specifically provides for the production of plants that further decrease lateral root formation under potassium deficient growth conditions relative to plants that have not been subjected to the methods of this invention. It is believed that the plants produced by these methods may thus display desirable agronomic or horticultural traits when grown under nutrient deficient conditions.

The method for obtaining a plant with decreased lateral root formation when grown under potassium deficient conditions comprises the steps of: (a) introducing an agent capable of reducing expression of at least one endogenous MYB77 gene in a plant cell; (b) regenerating a plant from said plant cell of step (a); and (c) identifying a regenerated plant from step (b) wherein said agent has reduced expression of said MYB77 gene in said regenerated plant by an amount sufficient to result in decreased lateral root formation when said regenerated plant is grown under potassium deficient conditions.

The introduced agent used in this method can be any one of:
i) a compound that effects an insertion, deletion, or point mutation in an endogenous MYB77 gene;
ii) ionizing radiation or ultraviolet light that effects an insertion, deletion, or point mutation in an endogenous MYB77 gene;
iii) a DNA fragment such as a T-DNA or transposon that is capable of insertion into a plant cell's genome (the T-DNA can be introduced into said plant cells with any one of Agrobacterium tumefaciens, Agrobacterium rhizogenes, Rhizobium, and Sinorhizobium);
iv) a transgene comprising any one of a fragment of the MYB77 gene, a sequence for producing a small interfering RNA (siRNA) capable of reducing expression of at least one endogenous MYB77 gene or a dominant negative mutant allele of said MYB77 gene. The dominant negative allele of the MYB77 gene may comprise a translational fusion of at least one DNA binding domain of the MYB77 gene and a transcriptional repressor domain, such as an Engrailed protein transcriptional repressor domain, an EAR protein transcriptional repressor domain, or an IAA/AUX domain I transcriptional repressor domain.

Identification of plants with reduced expression of the MYB77 gene can be accomplished by any number of techniques. For example, MYB77 gene expression can be assayed by methods such as binding assays to specific DNA sequences recognized by MYB77, an enzyme-linked immunoassays detecting MYB77 protein, RNA hybridization assays to detect MYB77 RNA, and assays employing reverse-transcriptase polymerase chain reaction directed to MYB77 sequences. It is noted herein that reduced expression of MYB77 may in some instances result in a decreased expression of a certain RNA sequence or protein sequences. For example, expression of auxin induced gene sequences or proteins, such as those for the AXR1, IAA19, IAA1, IAA28, SAUR-AC1, ARF7 and AUX1 genes, is shown herein to be decreased in plants with reduced MYB77 expression relative to wild-type plants upon auxin exposure. Consequently, plants or plant cells with reduced expression of MYB77 can be identified by assaying for reductions in expression of genes regulated by the MYB77 gene (i.e., AXR1, IAA19, IAA1, IAA28, SAUR-AC1, ARF7 and/or AUX1 genes) in auxin exposed plants by methods such as enzyme activity-based determination assays, assays that detect non-enzymatic biochemical activities associated with the proteins encoded by these genes, immunoassays, enzyme-linked immunoassays, RNA hybridization based assays, and reverse-transcriptase polymerase chain reaction-based assays.

Identification of plants with reduced expression of the MYB77 gene may also be confirmed by assaying a biological effect of this reduced expression. For example, plants subjected to these methods can be assayed for further decreases in lateral root formation relative to control plants (i.e., plants not subjected to the methods of the invention) when both plants are exposed to potassium deficient conditions to identify plants with reduced expression of the MYB77 gene.

This method is found to be useful in a variety of different types of plants when the expression of the appropriate MYB77 gene is reduced in that plant. When the plant is an Arabidopsis thaliana plant, the MYB77 gene may encode a cDNA sequence selected from the group of cDNA sequences that are essentially identical to SEQ ID NO:1. When the plant is a Zea mays plant, the MYB77 gene may encode a cDNA essentially identical to SEQ ID NO:2. When the plant is a Glycine max plant, the MYB77 gene encodes a cDNA essentially identical to SEQ ID NO:3. When the plant is a Gossypium hirsutum plant, the MYB77 gene encodes a cDNA essentially identical to SEQ ID NO:4. When the plant is an Oryzae species plant, the MYB77 gene encodes a cDNA essentially identical to SEQ ID NO:5.

### Plant Cells and Plants Produced by the Methods of the Invention

This invention also provides for a transgenic plant cell comprising a transcriptionally-competent AtMyb77 promoter fragment that is operably linked to a sequence of interest. A transcriptionally-competent AtMyb77 promoter fragment is a promoter fragment that can direct expression of an RNA and/or protein of an operably linked sequence of interest in an auxin-responsive plant root cell. The sequence of interest can be a reporter gene encoding a protein such as a beta-glucuronidase protein, a green fluorescent protein, a beta-galactosidase protein, a luciferase protein derived from a luc gene, a luciferase protein derived from a lux gene, a sialidase protein, streptomycin phosphotransferase protein, a nopaline synthase protein, an octopine synthase protein and a chloramphenicol acetyl transferase protein. The transcriptionally-competent AtMyb77 promoter fragment may comprise SEQ ID NO:6 or SEQ ID NO:7. The transcriptionally-competent AtMyb77 promoter fragment may also comprise a sequence that is at least 90% identical to SEQ ID NO:6 or SEQ ID NO:7. The plant cell may comprise a functional AtMyb77 promoter fragment operably linked to a beta-glucuronidase gene which is operably linked to a polyadenylation region as in SEQ ID NO:8 or SEQ ID NO:9. Furthermore, the sequence of interest can be a sequence capable of reducing a response to auxin in a plant root cell. The sequence capable of reducing a response to auxin in a plant root cell can be an antisense sequence, a silencing sequence, and a sequence encoding a small interfering RNA.

The invention also provides for a transgenic plant cell comprising a transgene that is capable of reducing expression of at least one endogenous MYB77 gene in said plant cell by an amount sufficient to reduce a response of said transgenic plant cell to auxin. This transgene may comprise a fragment of the MYB77 gene, a sequence for producing a small interfering RNA (siRNA) capable of reducing expression of at least one endogenous MYB77 gene, or a dominant negative mutant form allele of said MYB77 gene. The dominant negative allele of the MYB77 gene may comprise a translational fusion of at least one DNA binding domain of the MYB77 gene and an Engrailed protein transcriptional repressor domain.

The invention also provides for a transgenic plant cell comprising a transgene that is capable of reducing expression of an endogenous MYB77 gene by an amount sufficient to result in decreased lateral root formation in a plant grown under potassium deficient conditions. This transgene may comprise a fragment of the MYB77 gene, a small interfering RNA (siRNA) capable of reducing expression of at least one endogenous MYB77 gene, or a dominant negative mutant allele of said MYB77 gene. The dominant negative allele of the MYB77 gene may comprise a translational fusion of a DNA binding domain of the MYB77 gene and a transcriptional repressor domain, such as the Engrailed protein, Aux/IAA protein domain I or EAR protein transcriptional repressor domains.

### BRIEF DESCRIPTION OF THE DRAWINGS

FIG. 1. Arabidopsis plants overexpressing AtMyb77 (referred to herein as AtMYB77) show a phenotype similar to wild-type plants grown with IAA. (A) Wildtype Col-0 plants and AtMYB77 plants on medium without additional IAA and wildtype Col-0 plants on medium containing 1 μM IAA. (B) Northern blot analysis of AtMyb77 expression in Col-0 (1), AtMYB77 (2) atmyb77-1 (3) and Col-0 with 20 μM IAA (4). (C) AtMYB77 lines grown in soil also grow poorly and appear stunted compared to wildtype Col-0.

FIG. 2. The intensity and localization of the synthetic auxin regulated promoter DR5::GUS expression in Arabidopsis plants is altered by AtMyb77 expression. (A) Wildtype Col-0 expressing DR5::GUS. (B) atmyb77-1 T-DNA insertion line expressing DR5::GUS. (C) AtMYB77 line expressing DR5::GUS.

FIG. 3. Localization of ProAtMyb77::GUS activity in wild-type Arabidopsis roots. (A) Primary root tip (B) primary root zone where root hairs are present (C—H) progression of lateral root development, (I, J) expression in lateral roots after addition of 100 nM IAA for 24 hours. Each bar indicates 1 mm.

FIG. 4. Reduced AtMyb77 expression reduces lateral root growth sensitivity to exogenous auxin. (A) atmyb77-1 T-DNA insertion line and wild-type Arabidopsis Col-0 grown on MS plates with no added auxin have the same number and length of lateral roots. (B) atmyb77-1 has more lateral roots than wildtype Col-0 with 100 nM IAA and (C) 80 nM 2, 4-D. (D) Mean lateral root numbers per primary root under control conditions and with 100 nM IAA and 80 nM 2, 4-D (n=100±SE). Mean comparison (TUKEY HSD) was done and bars with different letters are significantly different (p<0.05).

FIG. 5. Lateral root growth is further decreased in the atmyb77 T-DNA insertion line under potassium and phosphorus deprived conditions and not under nitrogen deficiency. (A) Mean number of lateral roots per primary root (±SE) after 7 days of growth on Low salt medium plates containing full nutrients (+NPK), with low K+ (10 μM —K), with low (10 μM) phosphorus (—P) and with low (100 μM) nitrogen (—N). Promoter AtMyb77::GUS illustrates the down regulation of AtMyb77 in primary roots, lateral roots and shoots of plants grown with potassium (B, D, F) and deprived of potassium (C, E, G). Bar indicates 1 mm for B, C, D and E and 10 mm for F and G.

FIG. 6. Northern blots of Arabidopsis wildtype Col-0 and T-DNA insertion line atmyb77-1 roots grown in hydroponic conditions for 7 weeks and treated with 20 μM IAA for 0, 6, 30 and 54 hours. Northern blots were probed with 3′ probes from auxin regulated and related genes. Ribosomal RNA loading control shown at bottom.

FIG. 7. The AtMyb77 protein specifically binds to two different Myb binding sequences found in certain auxin regulated genes shown in FIG. 7. (A) AtMyb77 binds to oligonucleotides containing MRE (AACC) and MYBcore (CNGTTR) sequences and is out-competed by unlabeled competitor oligonucleotides 20 times in excess. (B) AtMyb77 does not bind to MRE (AGCC) or MYBcore (CNGTAR) when one nucleotide in the core sequence is changed nor does it bind to GCC (GCCGCC) which is found in ethylene responsive promoters.

FIG. 8. The plant shuttle vector pAKK1431 (SEQ ID NO:16).

FIG. 9. The plant transformation vector AKK1472B (SEQ ID NO:17).

### DETAILED DESCRIPTION OF THE INVENTION

### Definitions:

“MYB77 gene” refers to a gene in a plant that is functionally equivalent to the AtMyb77 gene of Arabidopsis thaliana. Plant MYB77 genes are defined as functionally equivalent to the AtMyb77 gene when they encode an R2R3-MYB-type DNA binding protein, are expressed in plant roots, and regulate the transcription of auxin-responsive genes in the plant roots.

When used to describe a nucleotide sequence of a plant gene or cDNA sequence, “essentially identical” refers to any of:
a) a nucleotide sequence of a gene or cDNA isolated from another variety, cultivar, or ecotype of the same plant species that is located at or is expressed from the same genetic locus (i.e., chromosomal location) and has the same function as the recited sequence;
b) a nucleotide sequence that encodes the same protein as the recited sequence; or
c) a nucleotide sequence that encodes a functionally indistinguishable protein.

When used herein to describe a protein sequence, “essentially identical” refers to a protein sequence of a protein that is functionally indistinguishable from the recited protein. Functionally indistinguishable proteins are typically characterized by the presence of one or more conservative amino acid substitutions.

When used herein to refer to a mutant form of a gene or its encoded protein, “dominant negative” refers to a derivative of a protein that blocks the activity of the wild-type or naturally occurring protein when the derivative is co-expressed with the wild type protein.

“Enzyme activity-based determination assays” refer to any assay that permits detection of a protein by measuring conversion of a substrate to a product by the protein.

As used herein, “biochemical activity-based assays” refer to any assay that permits detection of a protein by measuring any non-enzymatic biochemical activity associated with that protein.

As used herein, “non-enzymatic biochemical activities” are any measurable binding or transport activity associated with a protein that permit detection of that protein.

Non-limiting examples of non-enzymatic biochemical activities include:
i) binding of a protein to DNA, RNA, another protein, the same protein (i.e., as in dimerization, trimerization, or oligomerization), a lipid, a sugar, a polysaccharide, or a plant secondary metabolite (i.e., such as a sterol, an auxin, a cytokinin, ethylene, a carotenoid, a flavanoid, a triterpene, or a phenylpropanoid), an active site inhibitor, a suicide inhibitor or an allosteric inhibitor; and
ii) active or passive transport of any compound or ion across a biological membrane or phospholipid bilayer.

As used herein, “immunoassay” refers to any assay that permits detection of a protein based on the binding of an antibody that specifically binds that protein.

“Consensus sequence” refers to an amino acid, DNA or RNA sequence created by aligning two or more homologous sequences and deriving a new sequence that represents the common amino acid, DNA or RNA sequence.

“Homolog” refers to a gene related to a second gene by identity of either the DNA sequences or the encoded protein sequences. Genes that are homologs can be genes separated by the event of speciation (see “ortholog”). Genes that are homologs may also be genes separated by the event of genetic duplication (see “paralog”). Homologs can be from the same or a different organism and may perform the same biological function in either the same or a different organism.

“Orthologs” are two or more homologous genes in different species that evolved from a common ancestral gene by speciation. Orthologs may have the same biological function in different species.

“Paralogs” are two or more homologous genes in the same species that have diverged from each other as a consequence of genetic duplication. Paralogs may have overlapping or duplicative biological function in a species or may have biologically distinct functions.

“Percent identity” refers to the number of elements (i.e., amino acids or nucleotides) in a sequence that are identical within a defined length of two optimally aligned DNA, RNA or protein segments. To calculate the “percent identity”, the number of identical elements is divided by the total number of elements in the defined length of the aligned segments and multiplied by 100. When percentage of identity is used in reference to proteins it is understood that certain amino acid residues may not be identical but are nonetheless conservative amino acid substitutions that reflect substitutions of amino acid residues with similar chemical properties (e.g., acidic or basic, hydrophobic, hydrophilic, hydrogen bond donor or acceptor residues). Such substitutions may not change the functional properties of the molecule. Consequently, the percent identity of protein sequences can be increased to account for conservative substitutions.

“Regenerating a plant” refers to any method of obtaining a whole plant from any one of a seed, a plant cell, a group of plant cells, plant callus tissue, or an excised piece of a plant.

### Introduction of Agents Capable of Reducing Expression of Endogenous MYB77 Genes

To practice methods of this invention, it is first necessary to introduce an agent that is capable of reducing expression of an endogenous MYB77 gene. This agent can be a mutagen such as chemical compound, ultraviolet light, ionizing radiation, or an insertable DNA sequence such as a T-DNA or transposon. Chemical mutagens useful in the practice of this method include ethyl methanesulfonate (EMS), methylmethane sulfonate (MMS), N-ethyl-N-nitrosourea (ENU), triethylmelamine (TEM), a diepoxyalkane, 2-methoxy-6-chloro-9[3-(ethyl-2-chloro-ethyl)aminopropylamino]acridine dihydrochloride (ICR-170), nitrosoguanidine, N-methyl-N-nitrosourea (MNU), procarbazine, chlorambucil, cyclophosphamide, diethyl sulfate, acrylamide monomer, melphalan, nitrogen mustard, vincristine, dimethyinitosamine, N-methyl-N′-nitro-Nitrosoguanidine (MNNG), 7, 12 dimethylbenz(a)anthracene (DMBA), ethylene oxide, hexamethylphosphoramide-, bisulfan, 2-aminoguanidine, or formaldehyde. The mutagen may also be ultraviolet light or radiation such as gamma radiation or x-rays. Methods of treating plants with these mutagens are disclosed in U.S. Patent Application No. 20040053236, incorporated herein by reference in its entirety.

The T-DNA of Agrobacterium is also an insertional mutagen that can be used as an agent to reduce expression of an MYB77 gene. T-DNA mutagenesis methods have been described for plants such as Arabidopsis (Krysan et al., Plant Cell, 1999, 1: 2283-2290) and rice (Jeon et al., Plant J. June 2000;22(6):561-70).

A variety of transposons such as those in the Ac/Ds (Activator-Disassociation) family and the Enhancer-inhibitor system have been used to effect transposon mutagenesis in Arabidopsis and maize (Speulman et al. Plant Cell, Vol. 11, 1853-1866, October 1999; Das, L., and Martienssen, R, 1995, Plant Cell 7:287-294). It is similarly anticipated that such systems could also be used to effect transposon mutagenesis of endogenous MYB77 genes in various plant species.

Alternatively, the agent used to reduce expression of the endogenous MYB77 gene can be a transgene. A variety of transgenes can be used to reduce expression of the endogenous MYB77 gene. For example, transgenes containing fragments of an MYB77 gene in either the sense or the antisense orientation that are operably linked to a plant promoter can provide for silencing of an endogenous MYB77 gene. Such methods for effecting the silencing of endogenous plant genes are disclosed in U.S. Pat. No. 5,231,020, incorporated herein by reference in its entirety. It is noted that the MYB77 fragment need not have perfect identity to the endogenous MYB77 gene to effect reductions in MYB77 gene expression. In this regard, the MYB77 fragment in the transgene will typically have at least 80% sequence identity to the endogenous MYB77 gene. However, MYB77 transgene fragments with 90% or greater percent identity to the endogenous MYB77 gene are preferred. Although antisense or sense fragments of an MYB77 gene as small as 23 base pairs can be used in such constructs, MYB 77 sense or antisense fragments of 100 base pairs or more are preferred, and MYB77 sense or antisense constructs with more than 500 base pairs are most preferred. However, sense strand fragments used in the silencing constructs would be designed such that they are incapable of producing functional MYB77 protein. This can be accomplished by any number of strategies such as placing the sense strand fragment out of frame with any translation initiation codons in the primary transcript of the MYB77 transgene, using fragments of the MYB77 transgene that lack key functional domains, and/or incorporating translational stop codons in the MYB77 sequence. Without being limited by theory, it is further understood that the mechanism by which the transgene fragment may reduce expression of the endogenous MYB77 gene can be by any one of a transcriptional gene silencing mechanism, a post-transcriptional silencing mechanism, a mechanism involving small interfering RNA molecule production, and/or a mechanism involving RNA-directed DNA methylation.

Alternatively, plant expression vectors or transgenes designed to produce small interfering RNA (siRNA) directed against the MYB77 gene can be introduced into the plants or plant cells to reduce expression of the endogenous MYB77 gene. A variety of different transgene sequences for producing an siRNA are contemplated by this invention. The transgene sequence for producing a small interfering RNA (siRNA) may produce the siRNA either directly or may produce an RNA that results in the formation of an siRNA by the plant host. One non-limiting example of a sequence for producing an siRNA is described in U.S. Pat. No. 6,635,805, incorporated herein by reference in its entirety. U.S. Pat. No. 6,635,805 describes methods of silencing endogenous target plant genes with siRNA producing transgenes. These methods may employ transgenes comprising a promoter operably linked to DNA which can be transcribed in a plant cell. This RNA transcript in turn comprises an RNA plant virus sequence that can replicate in the cytoplasm of the plant cell. In general, the RNA transcript typically contains just those sequences required for its autonomous replication in the cytoplasm of the host cell. A key feature of the RNA transcript is the presence of at least one targeting sequence which causes post-transcriptional gene silencing of at least one target gene. This targeting sequence is foreign to the plant virus sequence, is 23 nucleotides or longer, and is at least 80% identical to the target sequence. In the case of this invention, the target sequence can be an MYB77 gene or a sequence that is at least 80% identical to the MYB77 gene sequence. In other embodiments of this invention, the target sequence can be a TIR1 gene, an AXR1 gene, an IAA19 gene, an IAA1 gene, an IAA28 gene, a SAUR-AC1 gene, a PIN1 gene, an ARF7 gene and an AUX1 gene.

Other methods of producing siRNA directed against a target gene are also contemplated by this invention. For example, a transgene sequence for producing an siRNA may comprise a promoter that is operatively linked to an intron encoding sequence and a hairpin RNA derived from a sequence in the target gene (Miki and Shimamoto, Plant Cell Physiol. April 2004;45(4):490-495.). Alternatively, a transgene sequence for producing an siRNA may comprise an RNA pol III promoter operably linked to a hairpin RNA (Lu et al., Nucleic Acids Res. December 2, 2004;32(21):e171.). The hairpin RNA may comprise a 5′ sequence of roughly 19-24 nucleotides of sense strand target gene sequence followed by a spacer nucleotide of about 8-10 nucleotides followed by a sequence of roughly 19-24 nucleotides of antisense sequence that is capable of base pairing with the preceding sense strand sequence. However, it has also been demonstrated that transgene sequences for producing hairpin RNA-expressing plant transgenes containing sense/anti-sense arms ranging from 98 to 853 nucleotides can yield efficient reductions in endogenous gene expression in a wide range of plant species (Wesley et al., Plant J. 2001, 27(6):581-90). Vectors and methods for effecting efficient inhibition of endogenous plant genes with transgene-mediated expression of hairpin RNAs are disclosed in U.S. Patent Application Nos. 20050164394, 20050160490, and 20040231016, each of which is incorporated herein by reference in their entirety.

Another type of transgene that can be used to reduce expression of an endogenous MYB77 gene is a transgene that encodes a dominant negative allele of the MYB77 protein. Dominant negative forms of the MYB77 protein may comprise either N-terminal, internal, or C-terminal truncations of the MYB77 that remove certain protein domains. For example, the DNA binding domains of MYB77 proteins are typically well conserved and easily identified in an MYB77 protein. For example, the Arabidopsis AtMyb77 protein (SEQ ID NO:10) contains two MYB DNA binding domains comprising amino acid residues 6-52 and 58-103 respectively of SEQ ID NO:10. Truncations of an MYB77 protein that retain one or both DNA binding domains of an MYB protein but remove other portions of the MYB77 protein that interact with transcription factors may function as dominant negative forms of MYB77. Without being limited by theory, forms of MYB77 that contain only a DNA binding domain may block productive binding of the intact endogenous MYB77 protein to the promoters of regulated genes and act as “dominant negatives”. Alternatively, truncations of a MYB77 protein that remove one or both DNA binding domains of a MYB protein but retain other portions of the MYB77 protein that interact with transcription factors may function as dominant negative forms of MYB77. Without being limited by theory, forms of MYB77 that do not contain a DNA binding domain may block productive interactions of the intact endogenous MYB77 protein with other transcription factors.

A specific type of dominant negative MYB77 form that can be useful in the practice of this invention comprise a DNA encoding translational fusion of at least one DNA binding domain of the Myb77 gene to an Engrailed protein transcriptional repressor domain. As described above, the DNA binding domains of MYB77 proteins are typically well conserved and easily identified in an MYB77 protein. For example, the Arabidopsis AtMyb77 protein (SEQ ID NO:10) contains two MYB DNA binding domains comprising amino acid residues 6-52 and 58-103 respectively of SEQ ID NO:10. One or both MYB77 DNA binding domains or the entire MYB77 coding sequence can be fused to a DNA sequence encoding an Engrailed protein transcriptional repression. Similar experiments with fusions of the engrailed repressor domain to other plant transcription factors have shown that Engrailed fusions provide for “dominant-negative” transgenes that phenocopy the effects of loss of function mutations in those transgenes (Markel et al. Nucleic Acids Research, 2002, Vol. 30, No. 21 4709-4719). The first 298 amino acids (SEQ ID NO:11) encoded by the Engrailed gene or any other DNA fragment may provide for transcriptional repression when fused to the N-terminus or C-terminus of complete or truncated plant transcription factors that include at least one DNA binding domain. Such fusions can be effected by in-frame fusions of the DNA fragment encoding the first 298 amino acids of the Engrailed protein to a second DNA fragment encoding the N-terminus or C-terminus of complete or truncated plant transcription factors that include at least one DNA binding domain.

Another specific type of dominant negative MYB77 form that can be useful in the practice of this invention comprises a DNA encoding a translational fusion of at least one DNA binding domain of the Myb77 gene and an EAR transcriptional repressor domain. As described above, the DNA binding domains of MYB77 proteins are typically well conserved and easily identified in an MYB77 protein. For example, the Arabidopsis AtMyb77 protein (SEQ ID NO:10) contains two MYB DNA binding domains comprising amino acid residues 6-52 and 58-103 respectively of SEQ ID NO:10. One or both MYB77 DNA binding domains or the entire MYB77 coding sequence can be fused to a DNA sequence encoding an EAR transcriptional repression domain. Similar experiments with fusions of the EAR repressor domain to other plant transcription factors including an MYB transcription factor AtMYB23 have shown that EAR fusions provide for “dominant-negative” transgenes that dominantly repress expression of the genes they ordinarily regulate (Hiratsu K, et al., Plant J. 2003;34(5):733-9). The EAR repressor domain comprising either a 12 amino acid sequence (SEQ ID NO:12; Hiratsu K, et al., Plant J. 2003;34(5):733-9) or 24 amino acid sequence (SEQ ID NO:13) of the Arabidopsis Ethylene-responsive transcription factor 4 (ERF4; Tiwari et al., The Plant Cell 16:533-543) may provide for transcriptional repression when fused to either the N-terminus or C-terminus of complete or truncated plant transcription factors that include at least one DNA binding domain. Finally it is further understood that any of the domain I transcriptional repressor regions of the Aux/IAA proteins (Tiwari et al., ibid) could also be fused to the MYB77 protein to obtain a dominant negative form of a MYB77 gene.

In the foregoing discussion of methods, it is understood that the MYB77 gene refers to a gene in a plant that is functionally equivalent to the AtMyb77 gene of Arabidopsis thaliana. Plant MYB77 genes that are functionally equivalent to the AtMyb77 gene in that they encode R2R3-MYB-type DNA binding proteins, are expressed in the plant's roots, and regulate the transcription of auxin-responsive genes in the plant's roots. Such MYB77 genes can be found in plant species from the genera Fabaceae, Medicago, Trifolium, Vigna, Daucus, Brassica, Raphanus, Sinapis, Lycopersicon, Capsicum, Solanum, Nicotiana, Helianthus, Bromus, Asparagus, Panicum, Pennisetum, Cucumis, Lolium, Glycine, Triticum, Gossypium, Oryza and Zea.

MYB77 genes can be identified in plant species other than Arabidopsis by employing either one or more methods familiar to those skilled in the art. One method is to identify such genes by performing analyses to determine the percent identity or percent similarity of a candidate MYB77 gene encoded protein to either the AtMyb77 protein sequence (SEQ ID NO:10) or a portion of the AtMyb77 protein sequence. This portion of the AtMYB77 sequence may comprise either one or both DNA binding domains (i.e., residues 6-52 and 58-103 respectively of SEQ ID NO:10) or regions from residues 104 to 304 of SEQ ID NO:10. The encoded proteins identified within the group of sequenced MYB genes of a given plant specie that have the highest percent identity or similarity to the AtMyb77 protein are candidate or potential MYB77 proteins (and corresponding MYB77 genes) of that plant species.

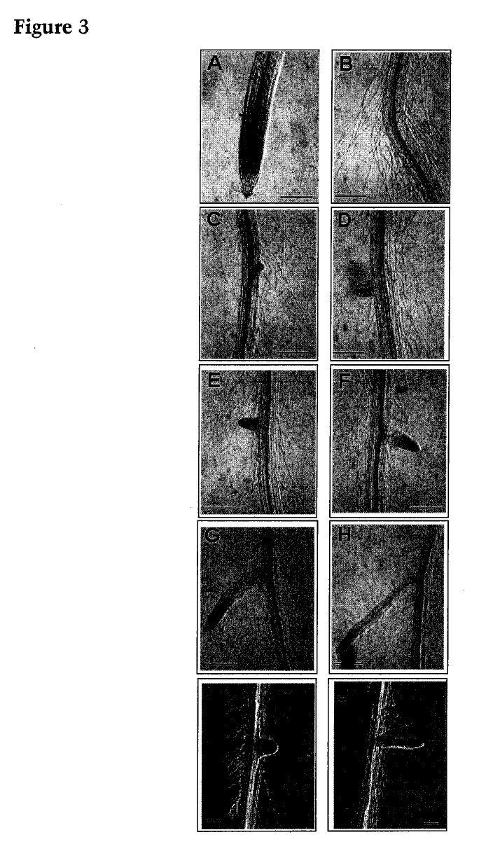

Having identified the MYB77 candidate genes in various crop plant species, several other criteria can be applied to confirm that the candidate MYB77 gene of a given crop plant species is functionally equivalent to the AtMyb77 gene. First, expression of the candidate MYB77 gene in plant roots can be confirmed by methods such as assays employing RNA hybridization or reverse-transcriptase polymerase chain reaction. These assays can be performed either on extracts of plant roots or on fixed and sectioned plant roots (i.e., in situ hybridization or PCR-based detection assays). Alternatively, the promoter of the endogenous MYB77 gene (i.e., a DNA fragment corresponding to about 2000 base pairs of 5′ flanking sequence and the entire 5′ untranslated portion of the MYB77 gene) can be fused to a reporter gene and assayed. It is anticipated that the MYB77 gene can be expressed in a spatial pattern corresponding to the spatial pattern disclosed here for the endogenous AtMyb77 promoter. The anticipated spatial pattern of expression can be: i) above and below the emerging lateral root primordia in the cortical cells (FIG. 3C); ii) in the lateral root above the tip immediately after lateral root emergence (FIGS. 3D and E); iii) in a discrete region just behind the tip at later stages of lateral root development (FIG. 3F, G, H); and iv) in the vascular tissue of the developing root (FIG. 3H). Furthermore, the addition of auxin (i.e., about 100 nM of IAA or the equivalent of another naturally occurring or synthetic auxin) may increase in the expression of the candidate MYB77 gene in regions beyond where the gene is normally expressed in the absence of added auxin, such as into the tip region. Finally, expression of the candidate MYB77 gene in roots may decrease upon growth in potassium deprived conditions (Shin and Schachtman, 2004).

Other useful criteria for identifying MYB77 proteins (and corresponding genes) as functional equivalents of the AtMyb77 proteins is the ability of those MYB77 proteins to regulate the expression of auxin-responsive genes in that crop plant. The ability of the plant MYB77 protein to specifically bind Myb binding sites present in the promoter regions of that plant's cognate IAA19, AUX1, SAUR-AC1, AXR1 and ARF7 genes (i.e., the promoters of the corn, soybean, cotton, and rice IAA19, AUX1, SAUR-AC1, AXR1 and ARF7 genes) is indicative of the functional equivalence of that MYB77 protein to the AtMyb77 protein.

However, the most critical criteria for confirming equivalence of the candidate MYB77 gene to AtMyb77 are functional criteria. More specifically, reduced expression of the functionally equivalent MYB77 gene in the crop plant specie is expected to result in:
i) a decreased response to auxin, such as decreased inhibition of lateral root formation upon auxin exposure relative to control plants with normal MYB77 expression; and
ii) further reductions in lateral root formation upon potassium or phosphate relative to control plants with normal MYB77 expression. Reduced expression of the MYB77 gene may also result in reduced expression of the crop plant's cognate AXR1, IAA19, IAA1, IAA28, SAUR-AC1, PIN1, ARF7 and/or AUX1 genes relative to control plants upon treatment of roots with auxin. Methods of reducing expression of endogenous MYB77 plants such as expression of MYB77 gene fragments or a small interfering RNA (siRNA) capable of reducing expression of the endogenous MYB77 gene with transgenes have been described herein. In particular, the use of siRNA methods is preferred as those skilled in the art are familiar with using such methods to rapidly assess the function of large numbers of genes in parallel (Wesley et al., Plant J. September 2001;27(6):581-90). In this regard, it is anticipated that multiple candidate MYB77 genes from a given crop plant can be tested in parallel to identify one or more MYB77 genes from that crop plant that are functionally equivalent to the AtMyb77 gene. Finally, methods of identifying plants with reduced MYB77 expression such as through use of DNA binding assays, RNA hybridization based assays, reverse-transcriptase polymerase chain reaction-based assays, and biological assays for auxin sensitivity or lateral root growth have also been described herein.

Any of the transgenes used for reducing the expression of an endogenous MYB77 gene can be introduced into the genome of a host plant via methods such as Agrobacterium mediated transformation, Rhizobium mediated transformation, Sinorhizobium mediated transformation, particle-mediated transformation, DNA transfection, DNA electroporation, or “whiskers”-mediated transformation. Aforementioned methods of introducing transgenes are well known to those skilled in the art and are described in U.S. Patent Application No. 20050289673 (Agrobacterium-mediated transformation of corn), U.S. Pat. No. 7,002,058 (Agrobacterium-mediated transformation of soybean), U.S. Pat. No. 6,365,807 (particle mediated transformation of rice), and U.S. Pat. No. 5,004,863 (Agrobacterium mediated transformation of cotton), each of which are incorporated herein by reference in their entirety. Methods of using bacteria such as Rhizobium or Sinorhizobium to transform plants is described in Broothaerts, et al., Nature. 2005,10;433(7026):629-33.

Transgenic plants are typically obtained by linking the gene of interest (i.e., in this case a transgene capable of reducing expression of a MYB77 gene) to a selectable marker gene, introducing the linked transgenes into a plant cell by any one of the methods described above, and regenerating the transgenic plant under conditions requiring expression of said selectable marker gene for plant growth. The selectable marker gene can be a gene encoding a neomycin phosphotransferase protein, a phosphinothricin acetyltransferase protein, a glyphosate resistant 5-enol-pyruvylshikimate-3-phosphate synthase (EPSPS) protein, a hygromycin phosphotransferase protein, a dihydropteroate synthase protein, a sulfonylurea insensitive acetolactate synthase protein, an atrazine insensitive Q protein, a nitrilase protein capable of degrading bromoxynil, a dehalogenase protein capable of degrading dalapon, a 2,4-dichlorophenoxyacetate monoxygenase protein, a methotrexate insensitive dihydrofolate reductase protein, and an aminoethylcysteine insensitive octopine synthase protein. The corresponding selective agents used in conjunction with each gene can be: neomycin (for neomycin phosphotransferase protein selection), phosphinotricin (for phosphinothricin acetyltransferase protein selection), glyphosate (for glyphosate resistant 5-enol-pyruvylshikimate-3-phosphate synthase (EPSPS) protein selection), hygromycin (for hygromycin phosphotransferase protein selection), sulfadiazine (for a dihydropteroate synthase protein selection), chlorsulfuron (for a sulfonylurea insensitive acetolactate synthase protein selection), atrazine (for an atrazine insensitive Q protein selection), bromoxinyl (for a nitrilase protein selection), dalapon (for a dehalogenase protein selection), 2,4-dichlorophenoxyacetic acid (for a 2,4-dichlorophenoxyacetate monoxygenase protein selection), methotrexate (for a methotrexate insensitive dihydrofolate reductase protein selection), or aminoethylcysteine (for an aminoethylcysteine insensitive octopine synthase protein selection).

Transgenic plants can also be obtained by linking a gene of interest (i.e., in this case a transgene capable of reducing expression of a MYB77 gene) to a scoreable marker gene, introducing the linked transgenes into a plant cell by any one of the methods described above, and regenerating the transgenic plants from transformed plant cells that test positive for expression of the scoreable marker gene. The scoreable marker gene can be a gene encoding a beta-glucuronidase protein, a green fluorescent protein, a beta-galactosidase protein, a luciferase protein derived from a luc gene, a luciferase protein derived from a lux gene, a sialidase protein, streptomycin phosphotransferase protein, a nopaline synthase protein, an octopine synthase protein or a chloramphenicol acetyl transferase protein.

The construction of transgenes that are expressed in monocotyledonous plants or dicotyledonous plants is well established. In general, such transgenes typically comprise a promoter that is operably linked to a sequence of interest which is operably linked to a polyadenylation or terminator region. In certain instances, such as in the expression of transgenes in monocot plants or in the expression of transgenes expressing siRNAs, it may also be useful to include an intronic sequence. When an intronic sequence is included it is typically placed in the 5′ untranslated leader region of the transgene. In certain instances, it may also be useful to incorporate specific 5′ untranslated sequences in a transgene to enhance transcript stability or to promote efficient translation of the transcript.

As used herein, the term “operably linked” means that a promoter is connected to a sequence of interest such that the transcription of that sequence of interest is controlled and regulated by that promoter. When the sequence of interest encodes a protein and when expression of that protein is desired, “operably linked” means that the promoter is linked to the sequence in such a way that the resulting transcript will be efficiently translated. If the linkage of the promoter to the coding sequence is a transcriptional fusion and expression of the encoded protein is desired, the linkage is made so that the first translational initiation codon in the resulting transcript is the initiation codon of the coding sequence. Alternatively, if the linkage of the promoter to the coding sequence is a translational fusion and expression of the encoded protein is desired, the linkage is made so that the first translational initiation codon contained in the 5′ untranslated sequence associated with the promoter and is linked such that the resulting translation product is in frame with the translational open reading frame that encodes the protein desired.

A variety of promoters can be used in the practice of this invention. For example, the promoter can be a viral promoter such as a CaMV35S or FMV35S promoter. The CaMV35S and FMV35S promoters are active in a variety of transformed plant tissues and most plant organs (e.g., callus, leaf, seed and root). Enhanced or duplicate versions of the CaMV35S and FMV35S promoters are particularly useful in the practice of this invention (U.S. Pat. No. 5,378,619, incorporated herein by reference in its entirety). Other useful nopaline synthase (NOS) and octopine synthase (OCS) promoters (which are carried on tumor-inducing plasmids of A. tumefaciens), the cauliflower mosaic virus (CaMV) 19S promoters, the light-inducible promoter from the small subunit of ribulose 1,5-bisphosphate carboxylase (ssRUBISCO, a very abundant plant polypeptide), the rice Act1 promoter and the Figwort Mosaic Virus (FMV) 35S promoter (see e.g., U.S. Pat. No. 5,463,175; incorporated herein by reference in its entirety). It is understood that this group of exemplary promoters is non-limiting and that one skilled in the art could employ other promoters that are not explicitly cited here in the practice of this invention.

It is also anticipated that root specific promoters can be particularly useful for driving expression of sequences of interest that are capable of reducing decreasing MYB77 gene expression since the MYB77 gene is ordinarily expressed in roots and certain trait alterations taught by this application are root associated traits. For example, U.S. Pat. No. 5,837,848, incorporated herein by reference in its entirety, discloses a root-specific promoter useful for expressing sequences of interest in the roots of monocot plants. Another potentially useful root-specific promoter shown to be active in both monocot and dicot plants is the PHT1 promoter that ordinarily regulates a phosphate transporter gene (Koyama et al., J Biosci Bioeng. January 2005;99(1):38-42). It is understood that these exemplary root-specific promoters are non-limiting and that one skilled in the art could employ other root specific promoters that are not explicitly cited here in the practice of this invention.

An intron may also be included in the DNA expression construct, especially in instances when the sequence of interest is to be expressed in monocot plants and/or if the sequence of interest is a hairpin RNA designed for production of siRNA. For monocot plant use, introns such as the maize hsp70 intron (U.S. Pat. No. 5,424,412; incorporated by reference herein in its entirety) or the rice Act1 intron (McElroy et al., 1990, The Plant Cell, Vol. 2, 163-171) can be used. Dicot plant introns that are useful include introns such as the CAT-1 intron (Cazzonnelli and Velten, Plant Molecular Biology Reporter 21: 271-280, September 2003), the PKANNIBAL intron (Wesley et al., Plant J. September 2001;27(6):581-90; Collier et al., 2005, Plant J 43: 449-457), the PIV2 intron (Mankin et al. (1997) Plant Mol. Biol. Rep. 15(2): 186-196) and the “Super Ubiquitin” intron (U.S. Pat. No. 6,596,925, incorporated herein by reference in its entirety; Collier et al., 2005, Plant J 43: 449-457) that have been operably integrated into transgenes. It is understood that this group of exemplary introns is non-limiting and that one skilled in the art could employ other introns that are not explicitly cited here in the practice of this invention.

As noted above, the sequence of interest may also be operably linked to a 3′ non-translated region containing a polyadenylation signal. This polyadenylation signal provides for the addition of a polyadenylate sequence to the 3′ end of the RNA. The Agrobacterium tumor-inducing (Ti) plasmid nopaline synthase (NOS) gene 3′ and the pea ssRUBISCO E9 gene (Fischhoff et al., 1987) 3′ un-translated regions contain polyadenylate signals and represent non-limiting examples of such 3′ untranslated regions that can be used in the practice of this invention. It is understood that this group of exemplary polyadenylation regions is non-limiting and that one skilled in the art could employ other polyadenylation that are not explicitly cited here in the practice of this invention.

Identification of Plants with Reduced MYB77 Expression

A variety of genetic screening schemes have been described that permit “reverse-genetic” identification of mutations in genes of interest. For example, U.S. Patent Application No. 20040053236, incorporated herein by reference in its entirety, describes methods for identifying point mutations in specific endogenous genes in populations of plants treated with chemical mutagens. This method, or variants thereof, would in this case comprise exposing plants or plant cells with a mutagen; isolating genomic DNA from the plants, plant cells or pools thereof; amplifying a segment of the MYB77 gene to produce a MYB77 amplification product; denaturing and reannealing the amplification product to produce heteroduplexes between the wild type and mutagenized MYB77 sequence; and identifying a point mutation in the MYB77 gene segment as compared to the sequence of the MYB77 gene in the parent organism or cell. A variety of methods of identifying heteroduplexes such as mismatch-specific chemical cleavage, electrophoretic detection (constant denaturant capillary electrophoresis (CDCE) or denaturing high pressure liquid chromatography (dHPLC)), or mismatch-specific enzymatic cleavage can be employed to identify plants carrying the mutation. Methods for detecting deletion mutants entailing use of polymerase chain reaction (PCR) conditions which favor the replication of truncated DNA strands over the synthesis of full length strands have also been described in U.S. Pat. No. 6,358,690, incorporated herein by reference in its entirety.

Methods for screening pools of plants mutagenized with a T-DNA to identify lines that contain insertions of the T-DNA in specific target genes have also been described (Young et al., Plant Physiol, 2001, 125: 513-518). In general, a pool of T-DNA mutagenized plants can be screened via use of polymerase chain reaction (PCR) employing DNA probes specific to MYB77 sequences and DNA probes to T-DNA specific sequences to identify lines containing T-DNA insertions in the MYB77 gene. Pools yielding positive PCR results can then be deconvoluted by performing additional rounds of PCR on subset pools and then upon individual plants to identify a line with a T-DNA insertion in the MYB77 gene. Detection or identification of transposon mutagenized MYB77 genes can be accomplished by similar PCR based techniques employing PCR primers specific to MYB77 sequences and transposon-specific sequences in screening of pools of mutagenized plants.

Another means of identifying a plant with reduced expression of an endogenous MYB77 gene is to directly assay plants subjected to agents that may reduce MYB77 expression for MYB77 expression. Expression of the endogenous MYB77 gene can be assessed by methods such as an assay measuring MYB77 DNA binding activity, an enzyme-linked immunoassay with antibodies that recognize the MYB77 protein, an RNA hybridization assay specific for MYB77 RNA, and an assay employing reverse-transcriptase polymerase chain reaction to detect MYB77 sequences assay. Alternatively, it is also taught herein that reduced expression of MYB77 proteins result in reduced expression of certain auxin responsive genes such as AXR1, IAA19, IAA1, IAA28, SAUR-AC1, PIN1, ARF7 and/or AUX1 in auxin-treated roots. Consequently one could assay plants subjected to agents that may reduce MYB77 expression for reduced expression of any, several or all of these auxin regulated genes (or their homologs in a crop plant such as corn, soybean, rice or cotton) to identify plants that may have reduced MYB77 expression. Reduced expression of MYB77 could then be confirmed in direct assays of MYB77 expression.

One skilled in the art will appreciate that any of the aforementioned methods of identifying a plant with reduced MYB77 expression could be applied to either the method for reducing a response of a plant cell to auxin of the instant invention or to the method for decreasing lateral root formation in a plant grown under potassium deficient conditions of the instant invention. Furthermore, one skilled in the art will also appreciate that the methods of identifying plants with reduced MYB77 expression disclosed herein could be applied in any order or in any combination in practicing the instant invention.

Certain biological assays for identifying plants with reduced MYB77 expression are also disclosed herein. For example, it is demonstrated herein that a plant with reduced MYB77 expression can display increased lateral root formation relative to control plants (i.e., plants not subjected to the methods of the invention) after auxin exposure. Consequently, one could assay plants subjected to agents that may reduce MYB77 expression for increased lateral root formation relative to control plants (i.e., plants not subjected to the methods of the invention) after auxin exposure to identify plants with reduced MYB77 expression. Reduced expression of MYB77 in the auxin-treated plants with increased lateral root formation relative to control plants could then be confirmed in direct assays of MYB77 expression. The auxin used can be a naturally occurring auxin such as indoleacetic acid, indolebutyric acid, indole-3-acetyl-myo-inositol ester, an esters thereof, or an amide amino acid conjugates thereof. The auxin used can be a synthetic auxin such as 2,4-dichloroacetic acid, naphthaleneacetic acid, clomeprop, 2,4,5-trichlorophenoxy acetic acid, 2,4-dichlorophenoxy butyric acid, dichlorprop, dichlorprop-P, monochlorophenoxy acetic acid, monochlorophenoxy acetic acid thioethyl, monochlorophenoxy butyric acid, mecoprop, mecoprop-P, chloramben, dicamba, 2,3,6-trichlorobenzoate, tricamba, quinchlorac, quinmerac, clopyralid, fluoroxypyr, picloram, trichlopyr, benazolin, chloramben, dicamba, an ester thereof, an amide thereof or a salt thereof. The synthetic auxin may also be 2-fluoroindole-3-acetic acid, 4-fluoroindole-3-acetic acid, 6-fluoroindole-3-acetic acid, 7-fluoroindole-3-acetic acid, 2-bromoindole-3-acetic acid, 4-bromoindole-3-acetic acid, 6-bromoindole-3-acetic acid, 7-bromoindole-3-acetic acid, 2-iodoindole-3-acetic acid, 4-iodoindole-3-acetic acid, 5-iodoindole-3-acetic acid, 6-iodoindole-3-acetic acid, 7-iodoindole-3-acetic acid, salts thereof, esters thereof, or an amide thereof. Finally, one skilled in the art would likely be able to devise other biologically based assays for decreased sensitivity to auxin, particularly in roots or lateral roots, that could be used to identify plants with reduced MYB77 expression.

The aforementioned biological assays can be particularly useful in that they provide methods both for obtaining plants that are resistant to auxin or herbicidal formulations of auxins and for obtaining auxin-resistant plants. Such assays are particularly useful in that they provide for direct identification of the desired auxin-resistance trait. However, any of the methods of identifying plants with reduced MYB77 expression disclosed herein can be used to obtain auxin resistant plants. The methods of this invention and the plants obtained by these methods can be resistant to an auxin-based herbicidal formulation applied by watering, spraying, atomizing, broadcasting, dusting, foaming, spreading, ultra-low volume method, treating soil, treating growth media and treating seeds. The methods of this invention and the plants obtained by these methods can be resistant to a naturally occurring auxin such as indoleacetic acid, indolebutyric acid, indole-3-acetyl-myo-inositol ester, an esters thereof, or an amide amino acid conjugates thereof. The methods of this invention and the plants obtained by these methods can be resistant to a synthetic auxin such as 2,4-dichloroacetic acid, naphthaleneacetic acid, clomeprop, 2,4,5-trichlorophenoxy acetic acid, 2,4-dichlorophenoxy butyric acid, dichlorprop, dichlorprop-P, monochlorophenoxy acetic acid, monochlorophenoxy acetic acid thioethyl, monochlorophenoxy butyric acid, mecoprop, mecoprop-P, chloramben, dicamba, 2,3,6-trichlorobenzoate, tricamba, quinchlorac, quinmerac, clopyralid, fluoroxypyr, picloram, trichlopyr, benazolin, chloramben, dicamba, an ester thereof, an amide thereof or a salt thereof. The methods of this invention and the plants obtained by these methods can be resistant to a synthetic auxin such as be 2-fluoroindole-3-acetic acid, 4-fluoroindole-3-acetic acid, 6-fluoroindole-3-acetic acid, 7-fluoroindole-3-acetic acid, 2-bromoindole-3-acetic acid, 4-bromoindole-3-acetic acid, 6-bromoindole-3-acetic acid, 7-bromoindole-3-acetic acid, 2-iodoindole-3-acetic acid, 4-iodoindole-3-acetic acid, 5-iodoindole-3-acetic acid, 6-iodoindole-3-acetic acid, 7-iodoindole-3-acetic acid, salts thereof, esters thereof, or an amide thereof.

Other biological assays for identifying plants with reduced MYB77 expression disclosed herein relate to the identification of plants that display decreased lateral root formation when grown under potassium deficient conditions. For example, plants subjected to an agent capable of decreasing expression of a MYB77 gene can be assayed for decreased lateral root formation relative to control plants under potassium deficient conditions. Those plants that display further reductions in lateral root formation relative to the control plants grown under the same potassium deficient conditions can then be assayed directly for reductions in MYB77 expression.

### Use of the AtMyb77 Promoter

The use of the AtMyb77 promoter in expressing sequences of interest in auxin responsive root cells is also contemplated by this invention. Given the applicants' discovery that this promoter is active in plant root cell types that respond to auxin, the AtMyb77 promoter is particularly useful for expressing genes that will modulate the growth of auxin responsive plant root cells. Moreover, operable fusions of the AtMyb77 promoter to reporter genes are useful in that they will permit determination of the effects of various agents used in agricultural on auxin responsive root cells. For example, the effect of various herbicides, safeners, insecticides, fungicides, soil treatments, and/or seed treatments on auxin responsive plant root cells can now be effectively monitored and optimized by use of the AtMyb77 promoter.

The construction of transgenes that typically comprise a promoter that is operably linked to a sequence of interest which is operably linked to a polyadenylation or terminator region has been previously described in this application. It is understood by those skilled in the art that the AtMyb77 promoter can be operably linked to any of the previously described gene expression elements such as 5′ untranslated leader regions, introns, and polydenylation sequences. It is understood that the named groups of exemplary intron or polyadenylation sequences is non-limiting and that one skilled in the art could employ other intron or polyadenylation sequences that are not explicitly cited here in the practice of this invention.

The methods of introducing transgenes and linked selectable and scoreable marker genes has also been described in this patent application. Methods of introducing the linked transgenes into a plant cell by methods such as Agrobacterium mediated transformation, Rhizobium mediated transformation, Sinorhizobium mediated transformation, particle-mediated transformation, DNA transfection, DNA electroporation, or “whiskers”-mediated transformation, and regenerating the transgenic plant under conditions requiring expression of a selectable marker gene for plant growth have also been described in this patent application. Methods for obtaining transgenic plants through use of scoreable marker genes have also been described in this patent application. It is understood that those descriptions would also apply to methods of using the AtMyb77 promoter.

When the sequence of interest is a coding region, it is further under stood that the AtMyb77 promoter can be operably linked to that coding sequence by either a transcriptional or translational promoter fusion. When the fusion is a translational fusion, the linkage is made so that the first translational initiation codon contained in the 5′ untranslated sequence is associated with the promoter and is linked such that the resulting translation product is in frame with the translational open reading frame that encodes the protein desired. In this case, the 5′ untranslated region can be the AtMyb77 5′ untranslated sequence and the initiator methionine can be the AtMyb77 initiator methionine as shown in SEQ ID NO:6. However, one skilled in the art will appreciate that additional codons derived from the AtMyb77 gene could be added at the N-terminus of such a translational fusion in instances where the linked coding sequence can tolerate addition of N-terminal amino acids. For example, between 1 to 40 amino acids from the N-terminus of the AtMyb77 coding region might be fused in frame to the open reading frame of an operably linked sequence of interest. An example of an AtMyb77 promoter sequence containing 1901 base pairs of promoter and 5′ untranslated sequence and the initiator methionine is shown in SEQ ID NO:6. An example of an operable translational fusion of the AtMyb77 promoter sequence containing 1901 base pairs of promoter and 5′ untranslated sequence and the initiator methionine to a sequence comprising a super-ubiquitin intron, a beta-glucuronidase gene, and a nopaline synthetase terminator is shown in SEQ ID NO:8.

The AtMyb77 promoter may also be linked to a coding or a non-coding sequence such as a hairpin RNA as a transcriptional fusion. An example of an AtMyb77 promoter sequence containing 1901 base pairs of promoter and 5′ untranslated sequence suitable for use in transcriptional fusions is shown in SEQ ID NO:7. If the transcriptional fusion is to be made to a coding sequence and expression of the encoded protein is desired, the linkage is made so that the first translational initiation codon in the resulting transcript is the initiation codon of the coding sequence. An example of an operable transcriptional fusion of the 1901 base pair AtMyb77 promoter and 5′ untranslated region to a sequence comprising a super-ubiquitin intron, a beta-glucuronidase gene, and a nopaline synthetase terminator is shown in SEQ ID NO:9.

Finally, one skilled in the art will further appreciate that derivatives of the AtMyb77 promoter sequence in addition to the particular AtMyb77 promoter fragments described herein can also be used in the practice of this invention. For example, the functional AtMyb77 promoter fragment used in this invention can comprise a sequence that is at is at least 90% identical to SEQ ID NO:6 or SEQ ID NO:7. Furthermore, subfragments of the functional AtMyb77 promoter fragments disclosed here as either SEQ ID NO:6 or SEQ ID NO:7 can also be used in the practice of this invention. Subfragments of SEQ ID NO:6 or SEQ ID NO:7 that can be used would at least comprise the final 200 nucleotides of the 3′ terminus of those sequences (i.e., nucleotides 1704 to 1904 of SEQ ID NO:6 or nucleotides 1701 to 1901 of SEQ ID NO:7). The 3′ terminus would be linked to the sequence of interest via an in-frame translational fusion in the case of the SEQ ID NO:6 subfragments. The 3′ terminus would be linked to the sequence of interest via a transcriptional fusion in the case of the SEQ ID NO:7 subfragments. The aforementioned subfragments of the SEQ ID NO:6 AtMyb77 promoter fragment can be between 200 to 1904 nucleotides in length while the aforementioned subfragments of the SEQ ID NO:7 AtMyb77 promoter fragment can be between 200 to 1901 nucleotides in length.

### EXAMPLES

The following examples illustrate the methods of the present invention and the preparation of plants and plant cells using these methods. The examples demonstrate certain methods and are not intended to limit the scope of the present invention. Those of skill in the art should, in light of the present disclosure, appreciate that many changes can be made in the specific embodiments which are disclosed and still obtain a like or similar result without departing from the spirit and scope of the invention.

### Example 1

### AtMyb77 Overexpression Mimics the Effects of Growth on Auxin

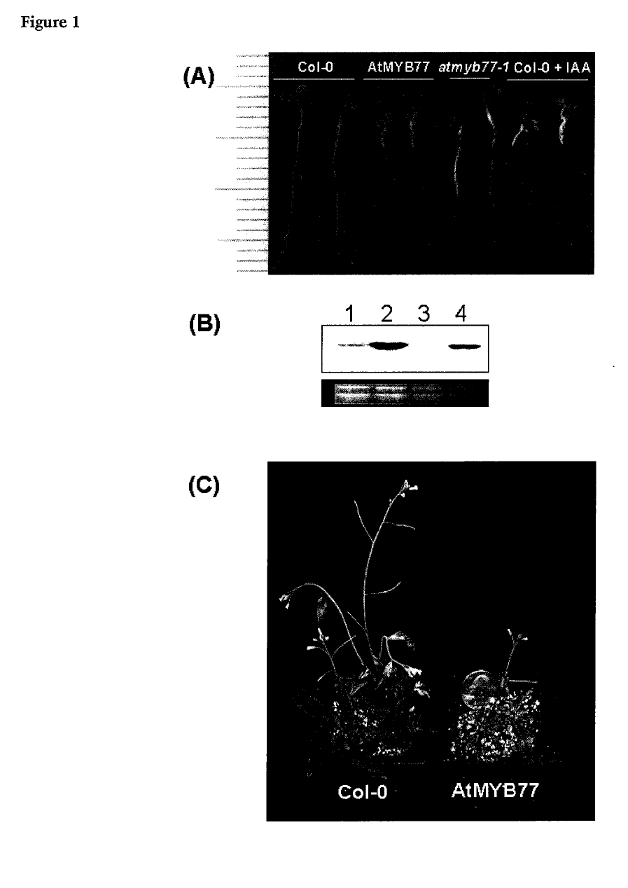

To determine the function of AtMyb77, the cDNA was over-expressed in Arabidopsis by operably linking the AtMyb77 gene to both the FMV promoter in pCambia2300 (Sanger et al. 1990) and to the Nopaline synthase terminator. Arabidopsis plants were transformed by Agrobacterium-mediated floral dip method (Clough and Bent 1998). Arabidopsis lines in which AtMyb77 was inactivated by a T-DNA insertion were obtained from the Arabidopsis Biological Research Center (Columbus, Ohio, USA) as “Salk_{—}67655”. The phenotypes of the over expressing and T-DNA insertion lines were characterized under control conditions. Strong visible phenotypes were observed in overexpression lines, but the T-DNA insertion lines with reduced AtMyb77 expression showed no visible phenotype under control conditions (FIGS. 1a and 1c). Stunted root and shoot growth was observed in the overexpression lines with an increased severity of stunting that was correlated to the level of overexpression (FIG. 1b). The stunted growth phenotype was reminiscent of wild-type plants grown on the plant hormone auxin (FIG. 1A).

### Example 2

### Expression of Genes that Respond to Auxin is Modulated by AtMyb77 Overexpression and in AtMyb77

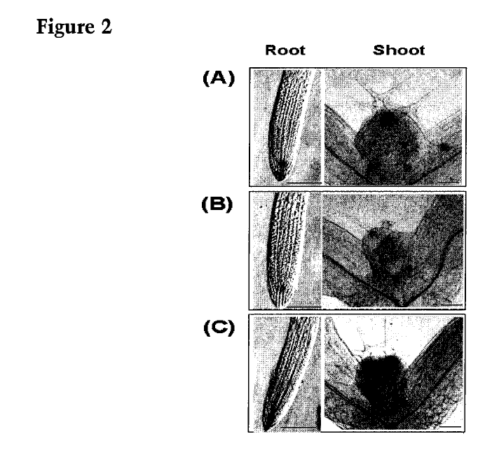

To initially test whether AtMyb77 is involved in auxin responses, transgenic Arabidopsis plants that overexpress the AtMyb77 protein and atmyb77-1 T-DNA insertion lines were crossed to plants containing the DR5::GUS synthetic promoter auxin responsive elements (Ulmasov et al. 1997). The wild type Arabidopsis expressing the DR5::GUS construct were incubated in X-GLUC solution to serve as a reference (FIG. 2a). In seedlings grown on control medium, GUS activity increased in roots and shoots of lines overexpressing AtMyb77 with the DR5::GUS marker (FIG. 2c). In contrast GUS activity was visibly reduced in roots and shoots of atmyb77-1 lines (i.e., the Arabidopsis line containing the T-DNA insertion in the AtMyb77 gene) that have reduced expression of the endogenous AtMyb77 gene (FIG. 2b). For quantification of GUS activity, the MUG assay was performed with homozygous lines that were grown for 4 days (Shin et al. 2003). These results indicate that an auxin response of a plant cell (i.e., expression of an auxin-regulated gene) can be upregulated by increasing the expression of AtMyb77. These results also indicate that an auxin response of a plant cell (i.e., expression of an auxin-regulated gene) can be down-regulated or reduced by reducing the expression of AtMyb77.

### Example 3

### Reporter Gene Expression Driven by an AtMyb77 Promoter

To identify an AtMyb77 promoter fragment useful for driving expression of sequences of interest in an auxin responsive cells, the gene expression was localized using the β-glucuronidase (GUS) reporter gene driven by the 1904 bp AtMyb77 promoter fragment and initiation codon (SEQ ID NO:6). This 1904 base pair AtMyb77 promoter fragment in SEQ ID NO:6 contains the translation initiation codon of the AtMyb77 gene, the entire 5′ untranslated region of the AtMyb77 gene and corresponding 5′ flanking region. This vector was constructed by first obtaining by PCR a 1918 Bp DNA fragment containing the AtMyb77 promoter fragment and a translation initiation codon (ATG) and cloning the PCR product into a PGEM-T Easy® vector (Promega, Madison, Wis.). The PCR primers used to obtain the AtMyb77 promoter from Arabidopsis genomic DNA were:

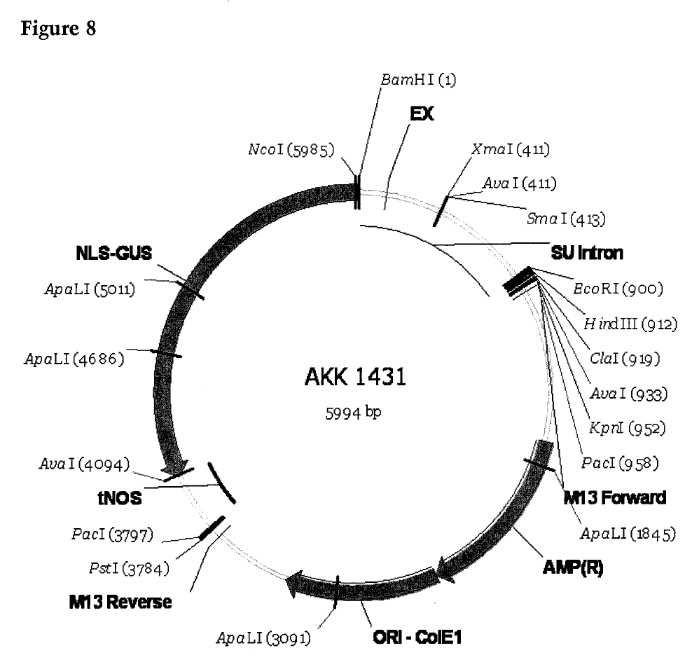

1) atMyb77 pro5′-kpnl: 5′ TGGTACCAAGGTTGGGAAGAAGGTGCAAATG-3′ (SEQ ID NO:14) and 2) atMyb77 pro3′-HindIII 5′-TAAGCTTCATTGAAACAGATCTTTTGTTTG-3′ (SEQ ID NO:15). The sequence of the AtMyb77 promoter fragment and a translation initiation codon (ATG) containing fragment was confirmed by DNA sequencing. The AtMyb77 promoter and ATG fragment in pGEM-T easy was isolated as a KpnI and HindIII fragment and cloned into the AKK1431 shuttle vector KpnI and HindIII sites (AKK1431 is shown in FIG. 8 and in SEQ ID NO:16). The resulting AtMyb77::GUS expression cassette comprises from 5′ to 3′ the 1901 Bp AtMyb77 promoter, an ATG transcriptional start codon of the AtMyb77 gene, a super-ubiquitin intron, a translational initiation site for the GUS gene, a translational termination site for the GUS gene, and a NOS 3′ terminator. Splicing of the super-ubiquitin intron places the AtMyb77 translational initiation codon in frame with the GUS or beta-glucuronidase gene.

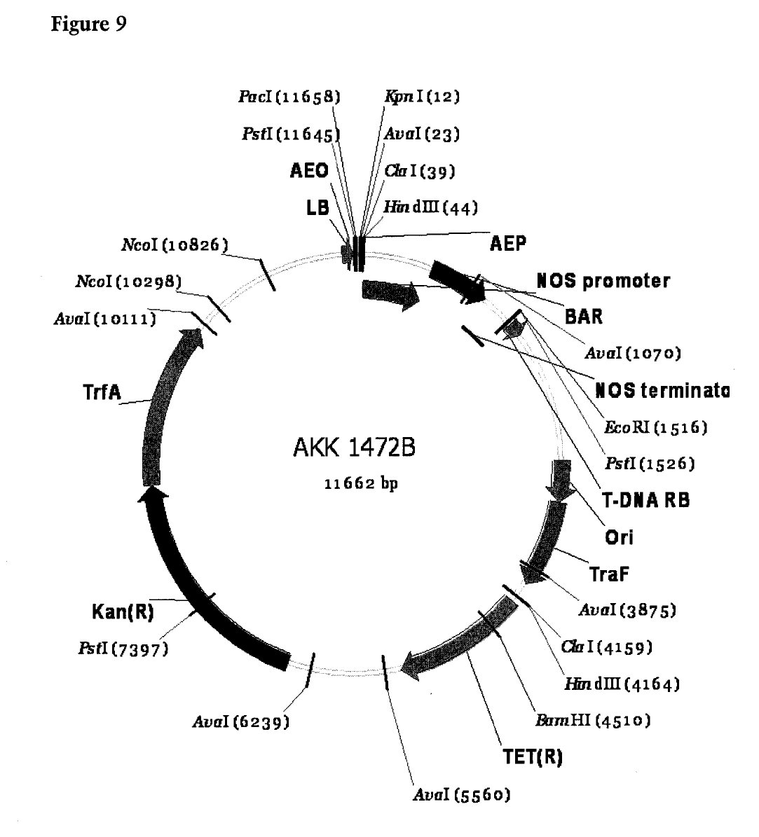

The AtMyb77 promoter::GUS-NOS fragment was cloned as a PacI fragment into AKK1472B (a plant transformation vector shown in FIG. 9 and SEQ ID NO:17). The binary vector constructs were introduced into the Agrobacterium tumefaciens strain C58C1::pGV3101. Arabidopsis plants were transformed by Agrobacterium-mediated floral dip method (Clough and Bent 1998).

To localize GUS activity, plants were stained with 1 mM X-Gluc solution 4 days after germination (DAG) (Vicente-Agullo et al. 2004) and were de-stained (Malamy and Benfey 1997). The pictures were taken using a Nikon SMZ1500 dissecting microscope (Nikon). Plants were transferred 3-DAG to full nutrient (1.75 mM KCl) or to low K plates (10 μM KCl), and then grown for 7 days before staining. For auxin treatment, plants were transferred 6-DAG to full nutrient plates (no auxin), and to plates containing 100 nM of IAA, or 80 nM of 2, 4-D, and then grown for one day before staining. Incubation time was 60 minutes and protein concentration was calculated with the CB-X kit (GenoTech).

Expression of AtMyb77-promoter driven GUS expression was detected in roots (FIG. 3) and leaves of seedlings. In roots the expression was localized to the inner cell layers of the primary root including the stele (FIGS. 3A and B). At the tip of primary roots expression of GUS was also seen in the root cap cells (FIG. 3A). Throughout the root expression was visible in the vascular tissue. An interesting pattern of expression was observed during the development of lateral roots. Initially when the lateral roots emerge, the reporter gene expression was observed above and below the emerging lateral root primordia in the cortical cells (FIG. 3C). As emergence of the lateral root progressed the expression of AtMyb77 promoter driven GUS expression became strongly localized in the lateral root above the tip (FIGS. 3D and E). As the lateral root developed, strong expression of AtMyb77 promoter driven GUS expression was seen in a discrete region just behind the tip (FIGS. 3F, G, H). As vascular tissue developed the expression of AtMyb77 could be seen in the vasculature (FIG. 3H). The addition of 100 nM of IAA to these AtMyb77 promoter::GUS plants caused an increase in the expression of this transcription factor in regions beyond where the gene is normally expressed such as into the tip region (FIGS. 3I and J). These results demonstrate that this AtMyb77 promoter fragment can provide for auxin induced expression of a linked sequence of interest.

### Example 4

### Reduced Expression of AtMyb77 Results in Reduced Auxin Sensitivity

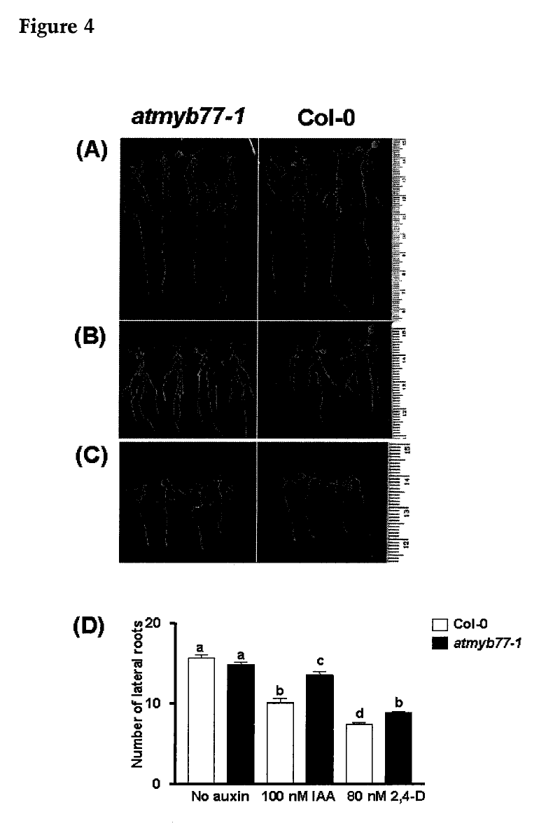

The effects of exogenous auxin on lateral root growth under control conditions were also tested in wild-type and atmyb77-1 plants. Wildtype Col-0 and atmyb77-1 plants had the same number of lateral roots and the same primary root length under control conditions (FIGS. 4A and D; Table I). However when Arabidopsis seedlings were treated with either 100 nM IAA (FIGS. 4B and D) or 80 nM 2,4-D (FIGS. 4C and D; Table I) lateral root formation was reduced significantly more in the wildtype as compared to the atmyb77-1 plants. Primary root length was the same between lines for each treatment. These results show that lateral root growth is less sensitive to the addition of exogenous auxin when AtMyb77 has been inactivated.

TABLE I
Comparison of lateral root number between wildtype (Col-0) and
atmyb77 T-DNA insertion lines (KO) with and without exogenous
auxin.

| | No hormone | 100 nM IAA | 80 nM 2,4,-D |
|---|---|---|---|
| Col-0 | 15.60 ± 0.41a | 10.05 ± 0.55b | 7.41 ± 0.20d |
| atmyb77 | 14.74 ± 0.35a | 13.57 ± 0.38c | 8.80 ± 0.19b |

Means within a column following by different letters indicates a difference at P < 0.01 (TUKEY HSD; n > 56 plants from 8 plates).

### Example 5

### Effects of Potassium Deprivation of AtMyb77 Promoter Expression and Effects of Reduced AtMyb77 Expression on Lateral Root Formation Under Potassium and Phosphorous Deficient Growth Conditions

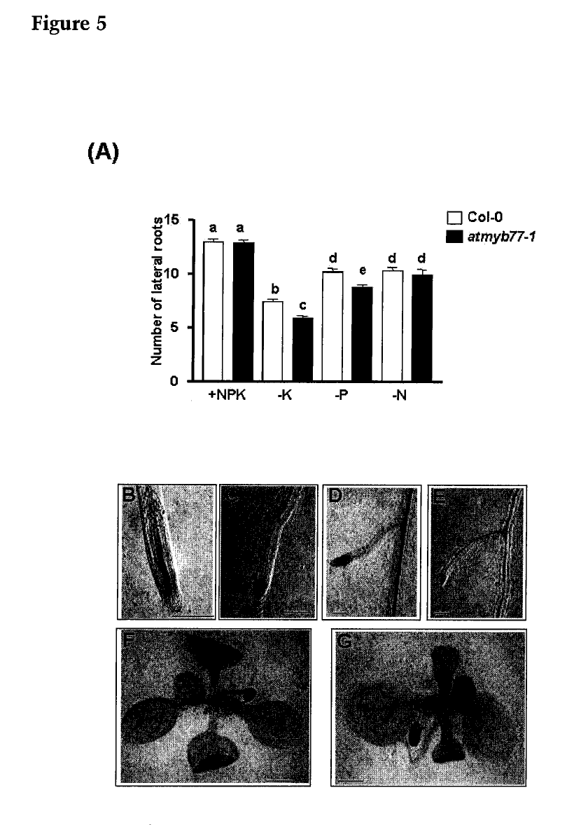

Expression of the AtMyb77::GUS expression cassette of SEQ ID NO:8 was first quantified under potassium (K+) deprived conditions (i.e., potassium deficient conditions). In wild-type plants that are deprived of K+, the expression of GUS was not detectable in leaves of the AtMyb77 promoter GUS plants (FIGS. 5F and G) and expression of GUS was reduced to low levels but not completely abolished in primary or lateral roots (FIGS. 5B-E). These results indicate that the AtMyb77 promoter of SEQ ID NO:6 is responsive to potassium deficits and can provide for reduced expression of a sequence of interest under conditions of potassium deprivation or reduction.

The effect of reducing AtMyb77 expression on lateral root formation under nutrient deprived conditions was also determined. For growth under normal conditions (i.e., nutrient rich conditions), media comprising 1.75 mM KCl and 0.5 M phosphoric acid was used. For growth under potassium deficient conditions, media comprising 10 μM KCl instead of 1.75 mM KCl was used. For growth under phosphate deficient conditions, media comprising 12 μM phosphoric acid instead of 0.5 M phosphoric acid was used. The number of lateral roots and the length of the primary root was the same for both the wildtype and the atmyb77-1 T-DNA insertion line under full nutrient conditions (FIG. 5A, Table II). However when plants were deprived of potassium or phosphorus, lateral root number in atmyb77-1 was significantly lower than the wild-type plants grown under the same potassium or phosphate deprived conditions (FIG. 5A, Table II). Primary root length was not different between lines under deprived conditions. Under nitrogen deprivation lateral root number and primary root length was the same for the wild-type and for atmyb77-1 plants. Under the conditions used in this study, lateral root number decreases in wild-type Col-0 under potassium deprivation are correlated with a decrease in expression of AtMyb77. A further significant decrease in lateral root number was measured in the atmyb77-T-DNA insertion line relative to the wild-type control when both the atmyb77-1 line and wild-type line are grown in parallel under reduced potassium or reduced phosphate conditions. Taken together, these data show that reducing AtMyb77 expression provides for reduced formation of lateral roots under either potassium or phosphate nutrient deficient conditions.

TABLE II
Comparison of lateral root number between wildtype (Col-0) and
atmyb77 T-DNA insertion lines (KO) under full nutrient (+NPK) and
nutrient deficient (−K, −P, and −N) condition.

| | +NPK | −K | −P | −N |
|---|---|---|---|---|
| Col-0 | 12.96 ± 0.25a | 7.44 ± 0.21b | 10.21 ± 0.30d | 10.33 ± |
| | | | | 0.32d |
| atmyb77 | 12.88 ± 0.25a | 5.83 ± 0.21c | 8.76 ± 0.23e | 9.92 ± 0.50d |

Means within a column following by different letters indicates a difference at P < 0.01 (TUKEY HSD; n > 48 plants from 8 plates).

### Example 6

### Reduced Expression of AtMyb77 Results in Reduced Expression of Auxin Regulated Genes in the Presence of Exogenous Auxin

To demonstrate the effect of reduced AtMyb77 expression on a variety of auxin induced genes, both wild-type and atmyb77-1 T-DNA insertion lines were grown in the absence and presence of auxin and assayed for expression of certain auxin induced genes (Berleth et al. 2004; Woodward and Bartel 2005). Plants were initially grown in nutrient solutions using a rock wool system (Gibeaut et al. 1997) for six weeks at 22° C. with 8 h daylight at 200 μmol.m-2.s-1. Three weeks after germination, plants were transferred to hydroponic tanks containing nutrient medium (Shin et al. 2005) with aeration. Plants were then exposed to a final concentration of 20 μM of IAA (Sigma-Aldrich, St.Louis, Mo.) in the nutrient solutions. After 6 h, 30 h and 54 h roots were harvested. For probes, cDNA fragments were amplified from Arabidopsis cDNA by PCR using primers for the indicated genes as follows:

IAA19,

(SEQ ID NO:18)

5′-CGAAAGTGGGGTTAGGGTATGTGAAAG.
and

(SEQ ID NO:19)

5′-TTTAACTCAACACTCAAGAAACAAGTAG;
AUX1,

(SEQ ID NO:20)

5′-ACGTTCGGATTCGCGTCTACACCG
and

(SEQ ID NO:21)

5′-TCATGGCTTGTGAGGAGGGCATTGG;
SAUR-AC1,

(SEQ ID NO:22)

5′-ATGGCTTTTTTGAGGAGTTTCTTG
and

(SEQ ID NO:23)

5′-TCATTGTATCTGAGATGTGACTGTG;
PIN1,

(SEQ ID NO:24)

5′-CAGGCTAAGG TGAT GCCACCAACAAG
and

(SEQ ID NO:25)

5′-TCATAGACCCAAGAGAATGTAGTAGAG;
AXR1,

(SEQ ID NO:26)

5′-CTTGTCATTGAGGAACGAGTTAAAAAC
and

(SEQ ID NO:27)

5′-ATGTAAGTCCCCAACATCGGAACAAAC;
ARF7,

(SEQ ID NO:28)

5′-TGCCTCTGGAACTTCTTACGGTTTAG
and

(SEQ ID NO:29)

5′-GGCTGAGTTATCAT CATAATGACCTC;
TIR1,

(SEQ ID NO:30)

5′-CTTCAACCTGCAAGGACCTACGCGAG
and

(SEQ ID NO:31)

5′-ATCC GTTAGTAGTAATGATTTGCCTGG;
IAA28,

(SEQ ID NO:32)

5′-TAACAGGGTTGAGGTAGCTCCAGTG GTG
and

(SEQ ID NO:33)

5′-CTATTCCTTGCCATGTTTTCTAGGTGAG;
IAA1,

(SEQ ID NO:34)

5′-ATGGAAGTCACCA ATGGGCTTAACC
and

(SEQ ID NO:35)

5′-TCATAAGGCAGTAGGAGCTTCGGATC.

The amplified cDNAs were labeled with α-³²P-dCTP (Amersham) and Northern blot analyses were performed as described (Shin et al. 2005).

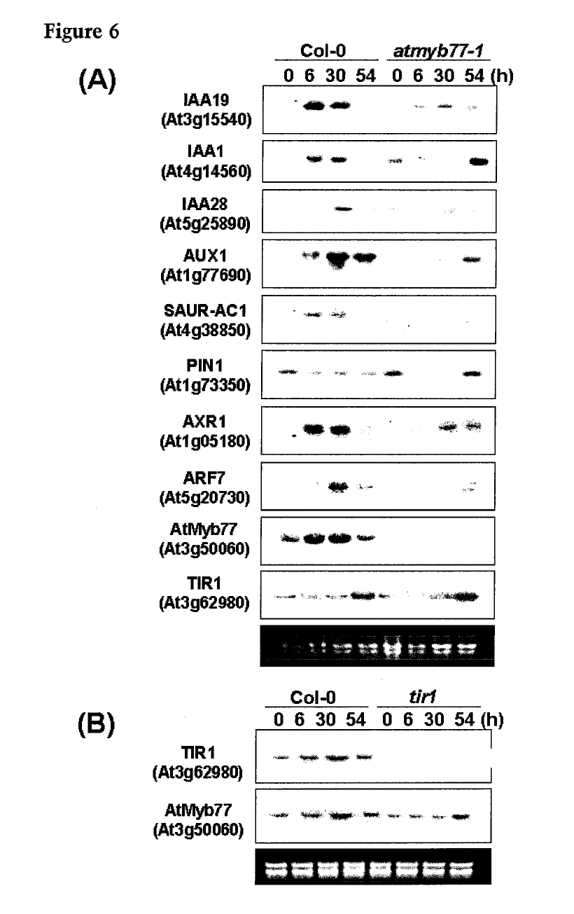

Clear induction of expression in the wild-type Arabidopsis was measured for IAA19, IAA1, IM28, AUX1, SAUR-AC1, AXR1, ARF7, AtMyb77 and Tir1 (comparison of 0 pre-auxin addition time point to 6, 30, and 54 hour time points in FIG. 6A). PIN1 expression was not induced by auxin in the wildtype after 54 hours. In contrast to the clear innduction of these genes in the wildtype, the induction was greatly attenuated or abolished in the T-DNA insertion atmyb77-1 T-DNA insertion line with reduced AtMyb77 expression. These results suggest that AtMyb77 is involved in modulating the expression of auxin responsive genes.

The expression of TIR1, which has been shown to be an auxin receptor (Kepinski and Leyser 2005), is regulated by the addition of exogenous auxin in roots and does not change significantly in the atmyb77-1 background. To determine whether AtMyb77 was downstream of TIR1, the seeds of tir1-1 were grown under the same hydroponic conditions and treated with 20 μM IAA and roots were harvested over a 54 hour period. TIR1 expression in the wildtype is induced in roots by auxin (Kepinski and Leyser 2005; Gray et al. 1999) whereas in the tir1-1 mutant the expression of TIR1 is greatly reduced (FIG. 6B). Unexpectedly the expression of AtMyb77 showed a similar response in the wildtype and in tir1-1 suggesting that AtMyb77 expression is regulated independently of the TIR1 receptor (FIG. 6B). These results also highlight the likelihood that other auxin receptors are present in Arabidopsis.

### Example 7

### Description of Additional AtMyb77 Promoter GUS Fusions

To identify an AtMyb77 promoter fragment useful for driving expression of sequences of interest in auxin responsive cells, the gene expression can be localized using the β-glucuronidase (GUS) reporter gene driven by the 1901 bp AtMyb77 promoter fragment (SEQ ID NO:7). This 1901 AtMyb77 promoter fragment in SEQ ID NO:7 contains the entire 5′ untranslated region of the AtMyb77 gene and corresponding 5′ flanking region. This vector can be constructed by first obtaining by PCR a DNA fragment containing the AtMyb77 promoter fragment and an translation initiation codon (ATG) and cloning the PCR product into a pGEM-T Easy® vector (Promega, Madison, Wis.). The PCR primers that can be used to obtain the AtMyb77 promoter from Arabidopsis genomic DNA are:

1) atMyb77 pro5′-kpnl: 5′ TGGTACCAAGGTTGGGAAGAAGGTGCAAATG-3′ (SEQ ID NO: 14) and 2) atMyb77 pro3′-HindIII 5′-TAAGCTTTGAAACAGATCTTTTGTTTGTGAAAC-3′ (SEQ ID NO: 36). The sequence of the AtMyb77 promoter fragment containing fragment can be confirmed by DNA sequencing. The AtMyb77 promoter and ATG fragment in pGEM-T easy can be isolated as a KpnI and HindIII fragment and then can be cloned into the AKK1431 shuttle vector KpnI and HindIII sites (AKK1431 is). The resulting AtMyb77::GUS expression cassette would comprise from 5′ to 3′ the 1901 Bp AtMyb77 promoter, an intron from small subunit of the RBC gene, a translational initiation site for the GUS gene, a translational termination site for the GUS gene, and a NOS 3′ terminator.

AKK1472B (plant expression vector) and AtMyb77 SEQ ID NO:7 promoter in AKK1431 can be digested with PacI. The AtMyb77 promoter::GUS fragment can then be cloned into AKK1472B (plant expression vector). The binary vector constructs can be introduced into the Agrobacterium tumefaciens strain C58C1::pGV3101. Arabidopsis plants can be transformed by Agrobacterium-mediated floral dip method (Clough and Bent 1998).

To localize GUS activity, plants can be stained with 1 mM X-Gluc solution 4 days after germination (DAG) (Vicente-Agullo et al. 2004) and were de-stained (Malamy and Benfey 1997). The pictures can be taken using a Nikon SMZ1500 dissecting microscope (Nikon). Plants can be transferred 3-DAG to full nutrient (1.75 mM KCl) or to low K plates (10 μM KCl), and then grown for 7 days before staining. For auxin treatment, plants can be transferred 6-DAG to full nutrient plates (no auxin), and to plates containing 100 nM of IAA, or 80 nM of 2, 4-D, and then grown for one day before staining.

For quantification of GUS activity, the MUG assay can be performed with homozygous lines that were grown for 4 days (Shin et al. 2003). Incubation time can be for 60 minutes and protein concentration can be calculated with the CB-X kit (GenoTech).

### Example 8

### Identification of Candidate MYB77 Genes from Crop Plant Species

To identify candidate MYB77 genes from crop plants, the 304? amino acid MYB77 sequence (SEQ ID NO:10) was used as a query sequence in a BLASTP search of both the monocot and dicot plant protein sequence data bases. The BLASTP search was performed using the BLASTP program v 2.2.10, Oct. 19, 2004 that was originally described by Altschul, S. F., et al., (1997), Nucleic Acids Res. 25:3389-3402. Queries were performed on Mar. 6, 2006 against the monocot and dicot plant genome databases maintained by Dr. Volker Brendel, Iowa State University, 1350 Beardshear hall Ames, Iowa 50011-0001 under the National Science foundation Award Number 0110254 at the internet address: http://plantgdb.org/PlantGDB-cgi/blast/PlantGDBblast. The MYB plant protein within a plant species displaying the highest percent identity of all the MYB proteins for that specie in the database was first identified. The corresponding gene that encodes the MYB proteins for that species in the database was then identified by accessing the nucleotide sequence identified in the annotation provided in the protein link. The thus identified MYB77 candidate genes for corn (SEQ ID NO:2), soybean (SEQ ID NO:3), cotton (SEQ ID NO:4), and rice (SEQ ID NO:5) are provided herein.

TABLE III
MYB77 Candidate Genes and Proteins from Arabidopsis, Corn,
Soybean, Cotton, and Rice

| | | | MYB77 Protein |
|---|---|---|---|
| | MYB77 Gene | MYB77 Gene | GenBank |
| Plant Species | SEQ ID: NO | GenBank GI. No. | Access. No. |
| Arabidopsis | SEQ ID NO: 1: | 2832405 | CAA74604.1 |
| thaliana | | | |
| Zea mays | SEQ ID NO: 2 | 19548466 | AAL90657 |
| Glycine max | SEQ ID NO: 3 | 5139813 | BAA81736 |
| Gossypium | SEQ ID NO: 4 | 13346187 | AAK19616 |
| hirsutum | | | |
| Oryzae sp. | SEQ ID NO: 5 | 50726432 | BAD34048 |

### Example 9

### Binding of the AtMyb77 Protein to Myb DNA Binding Motifs in Auxin Responsive Plant Gene Promoters

The promoter regions of five genes (IAA19, AUX1, SAUR-AC1, AXR1, and ARF7) that are induced by auxin treatment were analyzed to identify putative Myb transcription factor binding motifs (Table IV). Well known Myb binding motifs (Williams and Grotewold, 1997) were identified in all five of these promoters with multiple Myb bindings sites found in all. About 2000 bp upstream promoter region of each gene was analyzed through use of the analytical tools available at PlantCARE (http://intra.psb.ugent.be:8080/PlantCARE/index.html; Lescot, M et al. Nucleic Acids Research, 2002, Vol. 30, No. 1 325-327) and PLACE (http://www.dna.affrc.go.jp/PLACE/signalscan.html; K. Higo, Y. Ugawa, M. Iwamoto and T. Korenaga (1999) Plant cis-acting regulatory DNA elements (PLACE) database:Nucleic Acids Research Vol. 27 No. 1 pp. 297-300. The MRE (Hoerena et al. 1998) and MYBcore (Larsen 2003) binding sites were the most frequently and consistently found in all the promoters analyzed.

An AtMyb77 cDNA fragment was amplified by PCR and cloned into the Ndel and SacI sites of the pET28 vector (Novagen, Madison, Wis., USA). The primer set used for the amplification was 5′-CATATGATGGCGGATCGTGTTAAAGGTCCAT-3′ (SEQ ID NO:37) and 5′-GAGCTCCTCAACCTTAGGTGTTATTACTCCAC-3′ (SEQ ID NO:38). The resulting in-frame fusion was transformed into Escherichia coli strain BL21 (DE3). Overexpression of AtMyb77::His was induced by 0.5 mM isopropyl-β-D-thiogalactoside at 20° C. for 4 h. Bacteria were pelleted after the induction period, suspended in lysis buffer (50 mM NaH2PO4H2O, 300 mM NaCl, 10 mM Imidazole) and subjected to sonication on ice for 1 min. Bacterial lysates were then centrifuged. The His tagged fusion protein was recovered by affinity chromatography on Nickel-NTA (Sigma-Aldrich, St. Louis, Mo., USA) and the purified protein was used for gel mobility shift assays.

Both strands of the MRE element (AACCWACCAACCAACNGAACC; SEQ ID NO:39) (Hoerena et al. 1998), the MYBcore element (CAGTTGCGTTRCACGGTTACTGAC; SEQ ID NO:40) (Larsen 2003), mutated MRE (mMRE) element (AGCCWGCCAGCCAGCNGAGCC; SEQ ID NO:41), mutated MYBcore (mMYBcore) element (CAGTAGCGTARCACGGTAACTGAC; SEQ ID NO:42) and GCC box (ATAAAGAGCCGCCACTAAAATAAGCTT; SEQ ID NO:43; Fujimotoa et al., 2000) were synthesized and were end-labeled with α-32P-dATP (Amersham, Pittsburgh, Pa., USA) by T4 polynucleotide kinase (New England Biolabs, Beverly, Mass., USA) at 37° C. for 1 h. Each strand of labeled oligos was purified using QIAquick™ nucleotide removal kit (Qiagen, Valencia, Calif., USA) and annealed for 15 min. at RT for the double strands. The assay mixtures contained 100 ng of AtMyb77::His protein, 1 ng of labeled oligo, 1 μg of poly(dl-dC)·(dl-dC), 5 μg of BSA, binding buffer (4 mM Hepes-KOH, pH 7.9, 20 mM KCl, 0.2 mM EDTA, 4% glycerol, and 0.4 mM DTT) in a 20 μL reaction volume. The mixtures were incubated at room temperature for 30 min. and electrophoresed on a 10% polyacrylamide gel in 0.5× TBE buffer. The gel was dried, exposed to an imaging plate and scanned using Typhoon 9410 system (Amersham, Pittsburgh, Pa., USA).

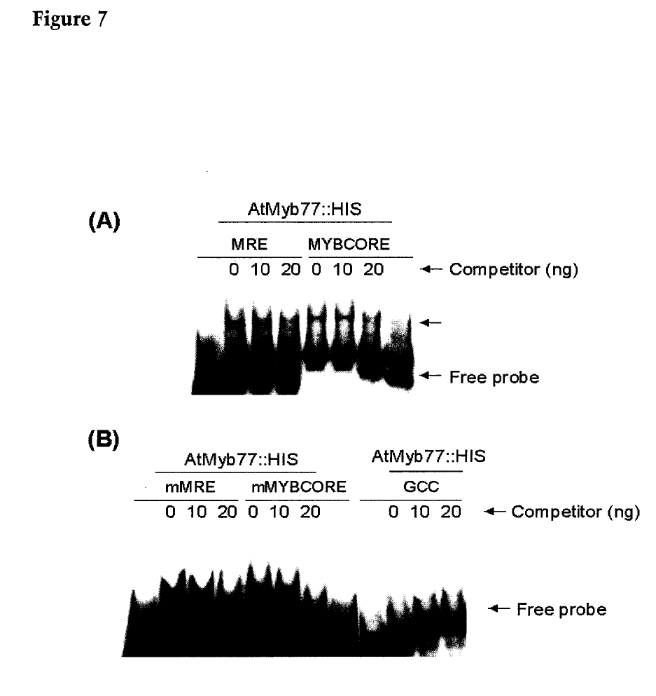

To test whether AtMyb77 bound to these MRE and MYBcore sequences identified in the auxin-responsive gene promoters, oligonucleotides were synthesized that contained multiple (3×) repeats of MRE and MYBcore and were labeled with ³²P. Band shift assays were performed using the HIS tagged AtMyb77 protein purified from E. coli in the presence and absence of unlabeled competitor oligonucleotides (FIG. 7A). AtMyb77 bound to both the MRE and MYBcore sequences and could be out competed with a 20-fold-excess of unlabeled oligonucleotides. To determine the specificity of binding in these assays, a single nucleotide change was made to the MRE and MYBcore oligonucleotdes (FIG. 7B). Changes of a single nucleotide eliminated the interaction and binding between MRE or MYBcore and AtMyb77. A known ethylene binding element was also tested (GCC) (Fujimoto et al. 2000) and no interaction between AtMyb77 was detected (FIG. 7B). This indicated that the binding of AtMyb77 protein to the MRE or MYBcore was specific.

TABLE IV
Numbers of different myb transcription factor binding sequences
found in auxin-regulated genes

| | Number of elements | | |
|---|---|---|---|
| | MRE | MYBcore | MBS |
| IAA19 | 6 | 3 | 3 |
| AUX1 | 4 | 4 | 0 |
| SAUR-AC1 | 3 | 2 | 1 |
| AXR1 | 4 | 6 | 0 |
| ARF7 | 6 | 3 | 0 |

Various publications are cited herein, the disclosures of which are incorporated by reference in their entireties.

### REFERENCES

Armengaud, P., R. Breitling, and A. Amtmann. 2004. The potassium-dependent transcriptome of Arabidopsis reveals a prominent role of jasmonic acid in nutrient signaling. Plant Physiol 136: 2556-2576.

Berleth, T., N. T. Krogan, and E. Scarpella. 2004. Auxin signals—turning genes on and turning cells around. Curr Opin Plant Biol 7:553-563.

Broothaerts W, Mitchell H J, Weir B, Kaines S, Smith L M, Yang W, Mayer J E, Roa-Rodriguez C, Jefferson R A. 2005. Gene transfer to plants by diverse species of bacteria. Nature. 2005. 433(7026):629-33.

Cazzonnelli, C. I. and J. Velten. 2003. Construction and Testing of an Intron-Containing Luciferase Reporter Gene From Renilla reniformis. Plant Molecular Biology Reporter 21: 271-280.

Clough, S. J. and A. F. Bent. 1998. Floral dip: a simplified method for Agrobacterium-mediated transformation of Arabidopsis thaliana. Plant J 16: 735-743.

Collier, R., B. Fuchs, N. Walter, W. K. Lutke, and C. G. Taylor. 2005. Ex vitro composite plants: an inexpensive, rapid method for root biology. Plant J 43: 449-457.

Cone, K. C., F. A. Burr, and B. Burr. 1986. Molecular analysis of the maize anthocyanin regulatory locus C1. Proc Natl Acad Sci U.S.A. 83:9631-9635.

Das, L., and Martienssen, R, 1995, Site-Selected Transposon Mutagenesis at the hcf706 Locus in Maize. Plant Cell 7:287-294.

Fujimotoa, S. Y., M. Ohtaa, A. Usuia, H. Shinshia, and M. Ohme-Takagia. 2000. Arabidopsis ethylene-responsive element binding factors act as transcriptional activators or repressors of GCC box-mediated gene expression. Plant Cell 12:393-404.

Ghosha, A. K., R. Steelea, and R. B. Ray. 1999. MBP-1 physically associates with histone acetylase for transcriptional repression. Biochem Biophysic Res Commun 260:405-409.

Gibeaut, D. M., J. Hulett, G. R. Cramer, and J. R. Seemann. 1997. Maximal biomass of Arabidopsis thaliana using simple, low-maintenance hydroponic method and favorable environmental conditions. Plant Physiol 115:317-319.

Gray, W. M., J. C. d. Pozo, L. Walker, L. Hobbie, E. Risseeuw, T. Banks, W. L. Crosby, M. Yang, H. Ma, and M. Estelle. 1999. Identification of an SCF ubiquitin-ligase complex required for auxin response in Arabidopsis thaliana. Gene Dev 13:1678-1691.

Higo, K., Y. Ugawa, M. Iwamoto and T. Korenaga (1999) Plant cis-acting regulatory DNA elements (PLACE) database: Nucleic Acids Research Vol. 27 No. 1 pp. 297-300.

Hiratsu K, K. Matsui, T. Koyama, and M. Ohme-Takagi (2003) Dominant repression of target genes by chimeric repressors that include the EAR motif, a repression domain, in Arabidopsis. Plant J. 2003;34(5):733-9

Hoerena, F. U., R. Dolferusa, Y. Wua, W. J. Peacock, and E. S. Dennis. 1998. Evidence for a role for AtMYB2 in the induction of the Arabidopsis alcohol dehydrogenase gene (ADH1) low oxygen. Genetics 149:479-490.

Jeon J S, Lee S, Jung K H, Jun S H, Jeong D H, Lee J, Kim C, Jang S, Yang K, Nam J, An K, Han M J, Sung R J, Choi H S, Yu J H, Choi J H, Cho S Y, Cha S S, Kim S I, An G. 2000. T-DNA insertional mutagenesis for functional genomics in rice. Plant J. 22(6):561-70

Kepinski, S. and O. Leyser. 2005. The Arabidopsis F-box protein TIR1 is an auxin receptor. Nature 435:446-451.

Koyama T, Ono T, Shimizu M, Jinbo T, Mizuno R, Tomita K, Mitsukawa N, Kawazu T, Kimura T, Ohmiya K, Sakka K. 2005. Promoter of Arabidopsis thaliana phosphate transporter gene drives root-specific expression of transgene in rice. J Biosci Bioeng. 99(1):38-42

Kranz, H. D., M. Denekamp, R. Greco, H. Jin, A. Leyva, R. C. Meissner, K. Petroni, A. Urzainqui, M. Bevan, C. Martin, S. Smeekens, C. Tonelli, J. Paz-Ares, and B. Weisshaar. 1998. Towards functional characterization of the members of the R2R3-MYB gene family from Arabidopsis thaliana. Plant J 16: 263-276.

Krysan, P. J, J. C. Young, and M. R. Sussman. 1999. T-DNA as an Insertional Mutagen in Arabidopsis. Plant Cell, 1: 2283-2290.

Larsen, K. 2003. Molecular cloning and characterization of a cDNA encoding a rye grass (Lolium perenne) ENOD40 homologue. J Plant Physiol 160:675-687.

Lescot M, Dehais P, Thijs G, Marchal K, Moreau Y, Van de Peer Y, Rouze P, Rombauts S. (2002) PlantCARE, a database of plant cis-acting regulatory elements and a portal to tools for in silico analysis of promoter sequences. Nucleic Acids Res. 30(1):325-7.

López-Bucio, J., A. Cruz-Ramirez, and L. Herrera-Estrella. 2003. The role of nutrient availability in regulating root architecture. Curr Opin Plant Biol 6:280-287.

Lee, M. and J. Schiefelbein. 1999. WEREWOLF, a MYB-related protein in Arabidopsis, is a position-dependent regulator of epidermal cell patterning. Cell 24:473-483.

Lu S, Shi R, Tsao C C, Yi X, Li L, Chiang V L. 2004. RNA silencing in plants by the expression of siRNA duplexes. Nucleic Acids Res. Dec. 2, 2004;32(21):e171.

Lym, R. and K. Moxness. 1989. Absorption, translocation, and metabolism of picloram and 2, 4-D in leafy spurge. Weed Sci 37:498-502.

Malamy, J. and P. Benfey. 1997. Organization and cell differentiation in lateral roots of Arabidopsis thaliana. Development 124:33-44.

Mankin, S. L, G. C. Allen, and W. F. Thompson. 1997. Introduction of a Plant Intron into the Luciferase Gene of Photinus Pyralis. Plant Mol Biol Rep 15(2): 186-196

Markel H, Chandler J, Werr W. 2002. Nucleic Acids Research. Translational fusions with the engrailed repressor domain efficiently convert plant transcription factors into dominant-negative functions. Nucleic Acids Res. 30(21): 4709-4719.

McCallum C M, Comai L, Greene E A, Henikoff S. 2000. Targeting induced local lesions IN genomes (TILLING) for plant functional genomics. Plant Physiol. 123(2):439-42.

McElroy, D, Zhang W, Cao J, Wu R. 1990. Isolation of an efficient actin promoter for use in rice transformation. The Plant Cell, Vol. 2, 163-171

Miki, D. and K. Shimamoto, 2004. Simple RNAi vectors for stable and transient suppression of gene function in rice. Plant Cell Physiol. 45(4):490-495

Montiel, G., P. Gantet, C. Jay-Allemand, and C. Breton. 2004. Transcription factor networks. Pathways to the knowledge of root development. Plant Physiol 136:3478-3485.

Nesi, N., C. Jond, I. Debeaujon, M. Caboche, and L. Lepiniec. 2001. The Arabidopsis TT2 gene encodes an R2R3 MYB domain protein that acts as a key determinant for proanthocyanidin accumulation in developing seed. Plant Cell 13:2099-2114.

Oppenheimer, D. G., P. L. Herman, S. Sivakumaran, J. Esch, and M. D. Marks. 1991. A myb gene required for leaf trichome differentiation in Arabidopsis is expressed in stipules. Cell 67:483-493.

Petroni, K., C. Tonelli, and J. Paz-Ares. 2002. The Myb transcription factor family: From maize to Arabidopsis. Maydica 47:213-232.

“Potassium Reduces Stress from Drought, Cool Soils and Compaction.” In Better Crops with Plant Food. Vol. 82 (1998, No. 3):34-36, published by the Potash and Phosphate Institute, Norcross, Ga.

Rehm, G. and Schmitt, M. 1997. Potassium for Crop Production. Publication 06794 of the University of Minnesota Extension Service.

Sanger, M., S. Daubert, and R. Goodman. 1990. Characteristics of a strong promoter from figwort mosaic virus: comparison with the analogous 35S promoter from cauliflower mosaic virus and the regulated mannopine synthase promoter. Plant Mol Biol 14:433-443.

Shin, R., R. H. Berg, and D. P. Schachtman. 2005. Reactive oxygen species and root hairs in Arabidopsis root response to nitrogen, phosphorus and potassium deficiency. Plant Cell Physiol 46:1350-1357.

Shin, R., M. Kim, and K.-H. Paek. 2003. The CaTin1 (Capsicum annum TMV-induced Clone1) and CaTin1-2 genes are linked head-to-head and share a bidirectional promoter. Plant Cell Physiol 44:549-554.

Shin, R. and D. P. Schachtman. 2004. Hydrogen peroxide mediates plant root response to nutrient deprivation. Proc Natl Acad Sci U.S.A. 101:8827-8832.

Speulman, E, Metz P L, van Arkel G, te Lintel Hekkert B, Stiekema W J, Pereira A. 1999. A two-component enhancer-inhibitor transposon mutagenesis system for functionalanalysis of the Arabidopsis genome. Plant Cell, Vol. 11, 1853-1866, October 1999;

Stracke, R., M. Werber, and B. Weisshaar. 2001. The R2R3-MYB gene family in Arabidopsis thaliana. Curr Opin Plant Biol 4:447-456.

Tatematsu, K., S. Kumagai, H. Muto, A. Sato, M. K. Watahiki, R. M. Harper, E. Liscum, and K. T. Yamamoto. 2004. MASSUGU2 encodes Aux/IA19, an auxin-regulated protein that functions together with the transcriptional activator NPH4/ARF7 to regulate differential growth responses of hypocotyl and formation of lateral roots in Arabidopsis thaliana. Plant Cell 16:379-393.

Tiwari S B, Hagen G, Guilfoyle T J .2004. Aux/IAA proteins contain a potent transcriptional repression domain. The Plant Cell 16:533-543

Ulmasov, T., J. Murfett, G. Hagen, and T. Guilfoyle. 1997. Aux/IAA proteins repress expression of reporter genes containing natural and highly active synthetic auxin response elements. Plant Cell 9:1963-1971.

Vicente-Agullo, F., S. Rigas, G. Desbrosses, L. Dolan, P. Hatzopoulos, and A. Grabov. 2004. Potassium carrier TRH1 is required for auxin transport in Arabidopsis roots. Plant J 40:523-535.

Weijers, D. and G. Jürgens. 2004. Funneling auxin action: specificity in signal transduction. Curr Opin Plant Biol 7:687-693.

Wesley S V, Helliwell C A, Smith N A, Wang M B, Rouse D T, Liu Q, Gooding P S, Singh S P, Abbott D, Stoutjesdijk P A, Robinson S P, Gleave A P, Green A G, Waterhouse P M. 2001. Construct design for efficient, effective and high-throughput gene silencing in plants. Plant J. 27(6):581-590.

Williams, C. E. and E. Grotewold. 1997. Differences between plant and animal Myb domains are fundamental for DNA binding activity, and chimeric Myb domains have novel DNA binding specificities. J Bio Chem 272:563-571.

Woodward, A. W. and B. Bartel. 2005. Auxin: Regulation, action, and interaction. Annal Bot 95:707-735.

Sample claims of various inventive aspects of the disclosed invention, not to be considered as exhaustive or limiting, all of which are fully described so as to satisfy the written description, enablement, and best mode requirement of the Patent Laws, are as follow:

What is claimed is:

1. A method for obtaining a plant with a decreased response to auxin, comprising the steps of:
a. introducing an agent capable of reducing expression of at least one endogenous MYB77 gene in a plant cell;
b. regenerating a plant from said plant cell of step (a); and
c. identifying a regenerated plant from step (b) wherein said agent has reduced expression of said MYB77 gene in said regenerated plant by an amount sufficient to decrease a response of said regenerated plant to auxin, thereby obtaining a plant with a decreased response to auxin.

2. The method of claim 1, wherein said agent of step (a) is a compound that effects an insertion, deletion, or point mutation in an endogenous MYB77 gene.

3. The method of claim 1, wherein step (a) comprises introduction of ionizing radiation or ultraviolet light to effect an insertion, deletion, or point mutation in an endogenous MYB77 gene.

4. The method of claim 1, wherein said agent is a DNA fragment capable of insertion into a plant cell's genome.

5. The method of claim 4, wherein said DNA fragment is a T-DNA.

6. The method of claim 5, wherein said T-DNA is introduced into said plant cells with a bacterium selected from the group consisting of Agrobacterium tumefaciens, Agrobacterium rhizogenes, Rhizobium, and Sinorhizobium.

7. The method of claim 4, wherein said DNA fragment is a transposon.

8. The method of claim 1, wherein said agent is a transgene comprising a fragment of said MYB77 gene.

9. The method of claim 8, wherein said transgene comprises a sequence for producing a small interfering RNA (siRNA) capable of reducing expression of at least one endogenous MYB77 gene.

10. The method of claim 1, wherein said agent is a transgene comprising a dominant negative mutant allele of said MYB77 gene.

11. The method of claim 10, wherein said dominant negative mutant allele of the MYB77 gene comprises a DNA encoding a translational fusion of at least one DNA binding domain of the MYB77 gene to either an Engrailed protein transcriptional repressor domain or an EAR transcriptional repressor domain.

12. The method of claim 1, wherein said auxin response is to auxin produced by said plant.

13. The method of claim 1, wherein said auxin response is to auxin produced by one or more cells of a whole plant.

14. The method of claim 1, wherein said auxin response is to auxin provided by an external source.

15. The method of claim 14, wherein said external source is a herbicidal formulation.

16. The method of claim 1, wherein the plant is an Arabidopsis thaliana plant and wherein said MYB77 gene encodes a cDNA sequence selected from the group of cDNA sequences that are essentially identical to SEQ ID NO:1.

17. The method of claim 1, wherein the plant is a Zea mays plant and wherein said MYB77 gene encodes a cDNA essentially identical to SEQ ID NO:2.

18. The method of claim 1, wherein the plant is a Glycine max plant and wherein said MYB77 gene encodes a cDNA essentially identical to SEQ ID NO:3.

19. The method of claim 1, wherein the plant is a Gossypium hirsutum plant and wherein said MYB77 gene encodes a cDNA essentially identical to SEQ ID NO:4.

20. The method of claim 1, wherein the plant cell is a Oryzae species plant and wherein said MYB77 gene encodes a cDNA essentially identical to SEQ ID NO:5.

21. The method of claim 1, wherein said response to auxin is inhibition of lateral root formation and wherein said auxin is provided by an external source.

22. A method for obtaining a transgenic plant expressing a sequence of interest in an auxin-responsive plant root cell, comprising the steps of:
a. constructing a DNA molecule comprising a functional AtMyb77 promoter fragment that is operably linked to said sequence of interest;
b. integrating said DNA molecule of step (a) into a plant cell's genome to obtain a transgenic plant cell;
c. regenerating a transgenic plant from said transgenic plant cell of step (b); and
d. confirming expression of said sequence of interest in an auxin-responsive plant cell of said transgenic plant of step (c), thereby obtaining a transgenic plant expressing a sequence of interest in an auxin-responsive plant root cell.

23. The method of claim 22, wherein said sequence of interest is a gene encoding a reporter protein that is operably linked to a polydenylation region.

24. The method of claim 23, wherein said reporter protein is selected from the group consisting of a beta-glucuronidase protein, a green fluorescent protein, a beta-galactosidase protein, a luciferase protein derived from a luc gene, a luciferase protein derived from a lux gene, a sialidase protein, streptomycin phosphotransferase protein, a nopaline synthase protein, an octopine synthase protein and a chloramphenicol acetyl transferase protein.

25. The method of claim 22, wherein said DNA molecule is SEQ ID NO:8 or SEQ ID NO:9.

26. The method of claim 22, wherein said functional AtMyb77 promoter fragment comprises SEQ ID NO:6 or SEQ ID NO:7.

27. The method of claim 22, wherein said sequence of interest is a sequence capable of reducing a response to auxin in a plant root cell.

28. The method of claim 27, wherein said sequence capable of reducing a response to auxin in a plant root cell comprises an antisense sequence, a silencing sequence, or a sequence for producing a small interfering RNA.

29. The method of claim 28, wherein said sequence capable of reducing a response to auxin in a cell is derived from a sequence encoding a protein selected from the group consisting of a TIR1 protein, an AXR1 protein, an IAA19 protein, an IAA1 protein, an IAA28 protein, a SAUR-AC1 protein, a PIN1 protein, an ARF7 protein and an AUX1 protein.

30. A method for obtaining a plant with decreased lateral root formation when grown under potassium deficient conditions, comprising the steps of:
(a) introducing an agent capable of reducing expression of at least one endogenous MYB77 gene in a plant cell;
(b) regenerating a plant from said plant cell of step (a); and
(c) identifying a regenerated plant from step (b) wherein said agent has reduced expression of said MYB77 gene in said regenerated plant by an amount sufficient to result in decreased lateral root formation when said regenerated plant is grown under potassium deficient conditions, thereby obtaining a plant with decreased lateral root formation when grown under potassium deficient conditions.

31. A transgenic plant cell comprising a functional AtMyb77 promoter fragment that is operably linked to a sequence of interest.

32. A transgenic plant cell comprising a transgene that is capable of reducing expression of at least one endogenous MYB77 gene in said plant cell by an amount sufficient to reduce a response of said transgenic plant cell to auxin.

33. A transgenic plant cell comprising a transgene that is capable of reducing expression of an endogenous MYB77 gene by an amount sufficient to result in decreased lateral root formation in a plant grown under potassium deficient conditions.
